(19) United States
(12) Patent Application Publication
(10) Pub. No.: US 2025/0049674 A1
(43) Pub. Date: Feb. 13, 2025

(54) COSMETIC OR DERMATOLOGICAL COMPOSITION COMPRISING AT LEAST A MEROCYANINE AND A HYDROTROPE

(71) Applicant: L'OREAL, Paris (FR)

(72) Inventors: Flavie GILLANT, CHEVILLY LA RUE (FR); Martin JOSSO, CHEVILLY LA RUE (FR); Manon DEMANGE, CHEVILLY LA RUE (FR); Marie-Lise CHIRON, CHEVILLY LA RUE (FR); Max DOMBROWSKI, Stuttgart (DE)

(21) Appl. No.: 18/720,237

(22) PCT Filed: Dec. 12, 2022
(86) PCT No.: PCT/EP2022/085439

§ 371 Date: Jun. 14, 2024

(30) Foreign Application Priority Data
Dec. 17, 2021 (FR) FR2113867

(51) Int. Cl. A61K 8/44; A61K 8/42; A61K 8/49; A61K 8/67; A61Q 17/04

(52) U.S. Cl. CPC A61K 8/44; A61K 8/42; A61K 8/4953; A61K 8/675; A61Q 17/04

(57) ABSTRACT

Cosmetic or dermatological composition comprising at least a merocyanine and a hydrotrope The present invention relates to a cosmetic or dermatological composition comprising an aqueous phase, and a merocyanine of formula (I):

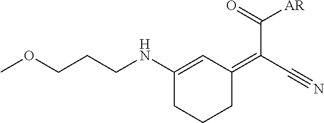

and at least one hydrotrope chosen from nicotinamide, caffeine, salicylic acid salts, the sodium salt of pyroglutamic acid (sodium PCA), sodium 1,3-benzenedisulfonate, sodium benzoate, sodium 4-pyridinecarboxylate, sodium benzenesulfonate, sodium p-toluenesulfonate (NaPTS), sodium butyl monoglycol sulfate (NaBMGS), 4-aminobenzoic acid HCl, sodium cumene sulfonate, N,N-diethyl nicotinamide, N-picolyl nicotinamide, N-allyl nicotinamide, 2-methacryloyloxyethyl phosphorylcholine, resorcinol, pyrogallol, N-picolylacetamide, procaine HCl, proline HCl, pyridine, 3-picolylamine, sodium ibuprofen, sodium xylene sulfonate (SXS), ethyl carbamate, pyridoxal hydrochloride, sodium benzoate, N,N-dimethylacetamide, N-methylacetamide, isoniazid, and mixtures thereof.

### TECHNICAL FIELD

The present invention relates to the field of caring for and/or making up keratin materials, in particular caring for keratin materials, notably the skin.

In particular, the present invention relates to the field of sun protection and more particularly relates to photoprotective cosmetic or dermatological compositions dedicated to the anti-UV protection of keratin materials which are endowed with advantageous properties in terms of sensory perception and stability.

For the purposes of the present invention, the term “keratin materials” notably denotes the skin, the lips and/or the eyelashes, in particular the skin and/or the lips, and preferably the skin of the body and/or the face, and more preferentially of the face.

### PRIOR ART

It is known that radiation with a wavelength of between 280 nm and 400 nm enables tanning of the human epidermis and that radiation with a wavelength of between 280 and 320 nm, known under the name UVB rays, harms the development of a natural tan. Exposure is also liable to bring about a detrimental change in the biomechanical properties of the epidermis, which is reflected by the appearance of wrinkles, leading to premature ageing of the skin.

It is also known that UVA rays with a wavelength of between 320 and 400 nm penetrate more deeply into the skin than UVB rays. UVA rays cause immediate and persistent tanning of the skin. Daily exposure to UVA rays, even of short duration, under normal conditions can result in damage to the collagen fibres and the elastin, which is reflected by a modification of the microrelief of the skin, the appearance of wrinkles and uneven pigmentation (liver spots, nonuniformity of the complexion).

Protection against UVA and UVB radiation is thus necessary. An effective photoprotective product must protect against both UVA and UVB radiation.

To date, a wide variety of photoprotective compositions have been proposed to overcome the effects induced by UVA and/or UVB radiation. They generally contain organic UV-screening agents and/or inorganic UV-screening agents, which function according to their own chemical nature and according to their own properties by absorption, reflection or scattering of the UV radiation. They generally contain mixtures of liposoluble organic screening agents and/or of water-soluble UV-screening agents combined with metal oxide pigments such as titanium dioxide or zinc oxide.

Many cosmetic compositions intended to limit the darkening of the skin, and to improve the colour and uniformity of the complexion have been proposed to date. It is well known in the field of antisun products that such compositions can be obtained by using UV-screening agents and in particular UVB-screening agents. Some compositions can also contain UVA-screening agents. This screening system must cover UVB protection, for the purpose of limiting and controlling the neosynthesis of melanin promoting overall pigmentation, but must also cover UVA protection, in order to limit and control the oxidation of the pre-existing melanin resulting in the darkening of the skin colour.

However, it is extremely difficult to find a composition containing a particular combination of UV-screening agents which would be especially suitable for the photoprotection of the skin and particularly for an improvement in the quality of the skin both in terms of the colour and in terms of its mechanical elasticity properties.

Advantageously, this improvement is particularly desired on skins which are already pigmented, for the purpose of not enhancing either the pigmentary melanin load or the structure of the melanin already present within the skin.

The majority of organic UV-screening agents consist of aromatic compounds which absorb in the range of wavelengths between 280 and 370 nm. In addition to their solar radiation-screening capacity, the desired photoprotective compounds must also have good cosmetic properties, good solubility in the usual solvents, and also good photostability alone or in combination with other UV-screening agents. They must also be colourless or at least have a colour which is cosmetically acceptable to the consumer.

One of the main disadvantages known to date of these compositions is that these screening systems have insufficient effectiveness against UV radiation and particularly against long UVA radiation with wavelengths above 370 nm with the aim of controlling light-induced pigmentation and the development thereof by a system which screens out UV radiation over the whole of the UV spectrum.

Among all the compounds that have been recommended for this purpose, an advantageous family of UV-screening agents which consists of carbon-bearing merocyanine derivatives has been proposed.

In particular, many cosmetic composition comprising UV-screening agents of merocyanine type are known in the prior art, for example in U.S. Pat. No. 4,195,999, application WO 2004/006878, document IP COM Journal 4 (4), 16 No. IPCOM000011179D published on 4 Mar. 2004, or else in application WO 2014/111570.

These compounds have very good screening properties in the long UVA radiation range. Moreover, the use of these screening agents in cosmetic products is considered to be safe, does not present acute toxicity, and does not induce eye irritation, skin sensitivity or genotoxicity.

Consequently, it has long been sought to formulate UV-screening agents of merocyanine type in the cosmetic and dermatological fields, in various galenic forms, due to its numerous beneficial properties, notably in large amount, for example of the order of 0.1% to 3% by weight.

However, these UV-screening agents do not have a very satisfactory solubility in the usual solvents, and have an unsatisfactory photostability for certain merocyanines.

With the aim of searching for other merocyanines which have better solubility in the usual solvents and better photostability, application WO 2013/011094 has proposed merocyanines comprising polar groups constituted of hydroxyl and ether functions, which show good long UVA-screening efficiency. However, the solubility of these particular merocyanines in oils is still not entirely satisfactory and often requires a tedious formulating process. Moreover, the large amounts of solvent that are required in order to dissolve this type of merocyanine may lead to cosmetic annoyances such as a tacky and greasy effect on application.

In addition, many cosmetic galenic forms, such as serums or aqueous gels, are devoid of a fatty phase. Thus, this solution does not make it possible to respond to the problem encountered with such compositions.

It is therefore necessary to be able to formulate UV-screening agents of merocyanine type in an aqueous medium. In particular, for cosmetic quality and performance benefits, it is advantageous that this aqueous medium be a single-phase medium. However, these screening agents prove to be very weakly soluble in such a medium. For example, the merocyanine (C) synthesised in example A4 below only has a water solubility of the order of 0.5 g/l at 20° C. which proves detrimental to the expression of its screening performance.

Consequently, it remains very difficult to use compounds of merocyanine type in aqueous phases, while retaining satisfactory sensory results, and with the lowest possible environmental impact.

### DISCLOSURE OF THE INVENTION

The aim of the present invention is to solve the abovementioned technical problems.

With regard to the above, it is thus clear that there remains a need to provide for consumers cosmetic and/or dermatological compositions comprising UV-screening agents of merocyanine type in the aqueous phase, while having satisfactory cosmetic properties, in particular as regards the sensory properties on application.

The need thus remains to improve the aqueous solubility of these merocyanines in photoprotective formulations, in particular over time.

In particular, there remains a need to increase the aqueous solubility of screening agents of merocyanine type by limiting the use of cosolvents that have an impact on the sensoriality.

The need thus remains for a cosmetic and/or dermatological composition, in particular photoprotective compositions, containing screening agents of merocyanine type in the aqueous phase, and which provide a good level of sensory pleasantness, whether this is during application on the skin or after drying.

Finally, there also remains a need to have compositions that are compatible with the current consumer demands, notably regarding the environment.

The present invention is specifically directed towards meeting these needs.

### SUMMARY OF THE INVENTION

Thus, according to a first aspect, the present invention relates to a cosmetic or dermatological composition, in particular for making up and/or caring for keratin materials, comprising at least an aqueous phase and:
at least one merocyanine of formula (I) below and also the E/E- or E/Z-geometric isomer forms thereof:

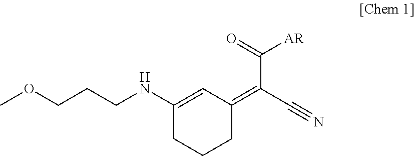

in which:
A is —O— or —NH;
R is a C₁-C₂₂ alkyl group, a C₂-C₂₂ alkenyl group, a C₂-C₂₂ alkynyl group, a C₃-C₂₂ cycloalkyl group or a C₃-C₂₂ cycloalkenyl group, it being possible for said groups to be interrupted by one or more 0; and
at least one hydrotrope chosen from nicotinamide, caffeine, salicylic acid salts, the sodium salt of pyroglutamic acid (sodium PCA), sodium 1,3-benzenedisulfonate, sodium benzoate, sodium 4-pyridinecarboxylate, sodium benzenesulfonate, sodium p-toluenesulfonate (NaPTS), sodium butyl monoglycol sulfate (NaBMGS), 4-aminobenzoic acid HCl, sodium cumene sulfonate, N,N-diethyl nicotinamide, N-picolyl nicotinamide, N-allyl nicotinamide, 2-methacryloyloxyethyl phosphorylcholine, resorcinol, pyrogallol, N-picolylacetamide, procaine HCl, proline HCl, pyridine, 3-picolylamine, sodium ibuprofen, sodium xylene sulfonate (SXS), ethyl carbamate, pyridoxal hydrochloride, sodium benzoate, N,N-dimethylacetamide, N-methylacetamide, isoniazid, and mixtures thereof.

The applicant has discovered, surprisingly, that by using particular hydrotropes, it is possible to improve the solubility of these merocyanines in an aqueous phase over time and at low temperature. This discovery forms the basis of the present invention.

In particular, the specific hydrotropes used in the compositions according to the invention make it possible to increase the aqueous ability of these merocyanines, and to prevent the phase separation of the composition containing them.

According to another of its aspects, the present invention also relates to a cosmetic process, in particular a non-therapeutic process, for caring for keratin materials, in particular of the body and/or of the face, comprising at least one step of applying a composition as defined above to said keratin materials.

Other features, aspects and advantages of the invention will become apparent on reading the detailed description which follows.

### DETAILED DESCRIPTION

A composition according to the invention is cosmetic and/or dermatological, and preferably is cosmetic.

A composition according to the invention is generally suitable for topical application to the skin and thus generally comprises a physiologically acceptable medium, i.e. a medium that is compatible with the skin.

It is preferably a cosmetically acceptable medium, i.e. a medium which has a pleasant colour, odour and feel and which does not cause any unacceptable discomfort, i.e. stinging, tautness or redness, liable to discourage the user from applying this composition.

### Merocyanines

As mentioned previously, a composition according to the invention comprises at least one merocyanine of formula (I) below, also the E/E- or E/Z-geometric isomer forms thereof:

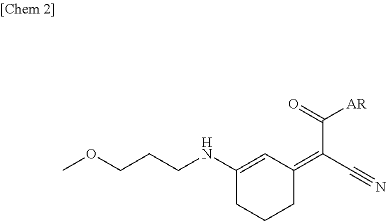

in which:
A is —O— or —NH;
R is a C₁-C₂₂ alkyl group, a C₂-C₂₂ alkenyl group, a C₂-C₂₂ alkynyl group, a C₃-C₂₂ cycloalkyl group or a C₃-C₂₂ cycloalkenyl group, it being possible for said groups to be interrupted by one or more O.

As indicated above, the merocyanine compounds of the invention can be in their E/E- or E/Z-geometric isomer forms:

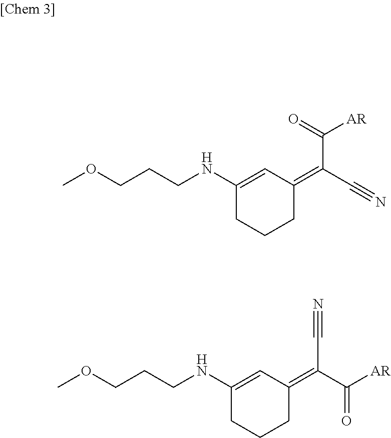

Preferably, in the compounds of formula (I), A is —O— and R is a C₁-C₂₂ alkyl which may be interrupted with one or more O.

Among the compounds of formula (I), use will more particularly be made of at least one compound chosen from the following compounds and also the E/E- or E/Z-geometric isomer forms thereof:

TABLE 1

| | |
|---|---|
| (A) | 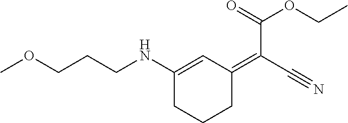 [SMILES: CCOC(=O)/C(C#N)=C1\C=C(NCCCOC)CCC1] |
| | ethyl (2Z)-cyano{3-[(3- |
| | methoxypropyl)amino]cyclohex-2-en- |
| | 1-ylidene}ethanoate |
| (B) | 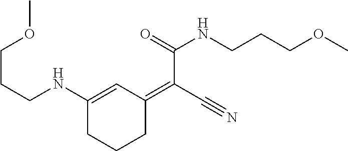 [SMILES: COCCCNC(=O)/C(C#N)=C1\C=C(NCCCOC)CCC1] |
| | (2Z)-2-cyano-N-(3-methoxypropyl)-2- |
| | {3-[(3-methoxypropyl)amino]cyclohex- |
| | 2-en-1-ylidene}ethanamide |
| (C) | 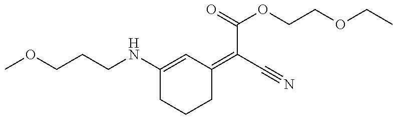 [SMILES: CCOCCOC(=O)/C(C#N)=C1\C=C(NCCCOC)CCC1] |
| | 2-ethoxyethyl (2Z)-cyano{3-[(3- |
| | methoxypropyl)amino]cyclohex-2-en- |
| | 1-ylidene}ethanoate |
| (D) | 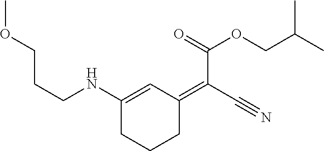 [SMILES: COCCCNC1=C/C(=C(/C#N)C(=O)OCC(C)C)CCC1] |
| | 2-methylpropyl (2Z)-cyano{3-[(3- |
| | methoxypropyl)amino]cyclohex-2-en- |
| | 1-ylidene}ethanoate |
| (E) | 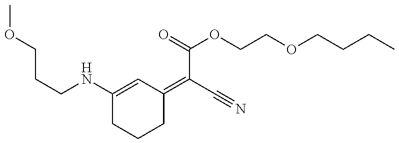 [SMILES: CCCCOCCOC(=O)/C(C#N)=C1\C=C(NCCCOC)CCC1] |
| | 2-butoxyethyl (2Z)-cyano{3-[(3- |
| | methoxypropyl)amino]cyclohex-2-en- |
| | 1-ylidene}ethanoate |
| (F) | 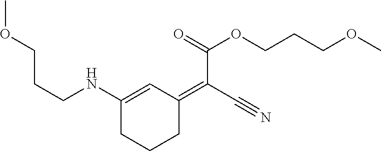 [SMILES: COCCCNC1=C/C(=C(/C#N)C(=O)OCCCOC)CCC1] |
| | 3-methoxypropyl (2Z)-cyano{3-[(3- |
| | methoxypropyl)amino]cyclohex-2-en- |
| | 1-ylidene}ethanoate |
| (G) | 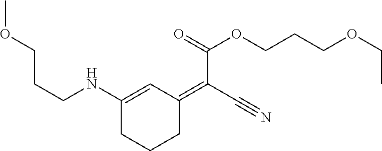 [SMILES: CCOCCCOC(=O)/C(C#N)=C1\C=C(NCCCOC)CCC1] |
| | 3-ethoxypropyl (2Z)-cyano{3-[(3- |
| | methoxypropyl)amino]cyclohex-2-en- |
| | 1-ylidene}ethanoate |

According to a more particularly preferred mode of the invention, use will be made of the compounds (A) and/or (C) and mixtures thereof, and even more preferentially the compound (C) in its E/E and/or E/Z geometric configuration.

Thus, preferably, a composition according to the invention comprises 2-ethoxyethyl (2Z)-cyano{3-[(3-methoxypropyl)amino]cyclohex-2-en-1-ylidene}ethanoate (C), also known as Methoxypropylamino Cyclohexenylidene Ethoxyethylcyanoacetate, in its E/E and/or E/Z geometric configuration.

The E/Z form has the following structure:

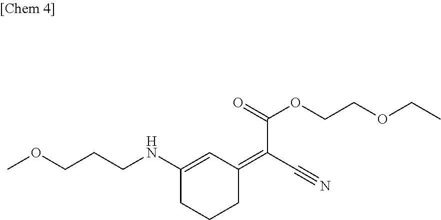 [SMILES: CCOCCOC(=O)/C(C#N)=C1\C=C(NCCCOC)CCC1]

The E/E form has the following structure:

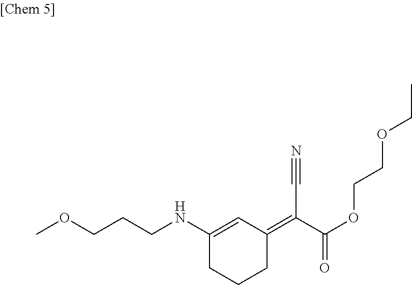 [SMILES: CCOCCOC(=O)/C(C#N)=C1/C=C(NCCCOC)CCC1]

According to a preferred embodiment, a composition according to the invention comprises at least one merocyanine of formula (I) as defined above, in a content ranging from 0.1% to 25% by weight, preferentially from 0.2% to 20% by weight, more preferentially from 0.5% to 10% by weight, and even better still from 1% to 5% by weight, relative to the total weight of the composition.

Generally, the compounds of merocyanine type can be prepared according to known processes, as described for example in J. Org. Chem. USSR (English Translation) 26(8), p. 1562f (1990); J. Heterocycl. Chem. 33(3), pages 763-766 (1996); Khimiya Geterotsiklicheskikh Soedinenii 11, pages 1537-1543 (1984); Khimiya Geterotsiklicheskikh Soedinenii 3, pages 397-404 (1982); Chem. Heterocycl. Comp. (English Translation), 24(8), 914-919 (1988), and in Synthetic Communications, Vol. 33, No. 3, 2003, pages 367-371.

As regards compounds of formula (I) more particularly considered according to the invention, and which have the specific feature of possessing a carbocyclic ring containing 6 carbon atoms, they may be prepared according to the protocols described in patent application WO 2007/071582, in IP.com Journal (2009), 9(5A), 29-30 IPCOM000182396D under the title “Process for producing 3-amino-2-cyclohexan-1-ylidene compounds” and in U.S. Pat. No. 4,749,643 on col. 13, line 66-col. 14, line 57, and the references cited in this regard.

In particular, compounds of formula (I) from Table 1 above may be synthesised according to the synthesis scheme described in the publication by B. Winkler et al., Tetrahedron Letters, 55 (2014) 1749-1751, which is entitled “A cyclic merocyanine UV-A absorber: mechanism of formation and crystal structure”, and is represented below, for the compounds of formula (I):

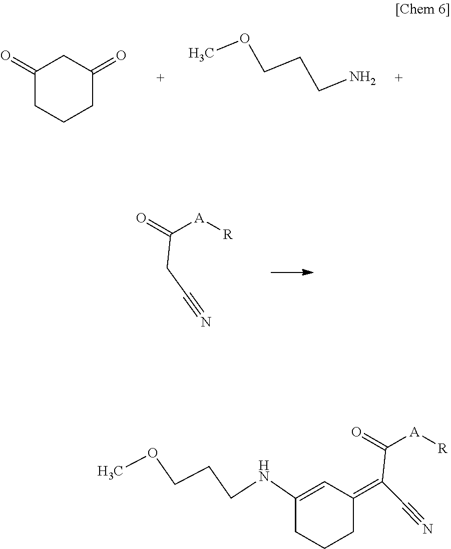

As regards compound (C) from Table 1, the following synthesis scheme is favoured.

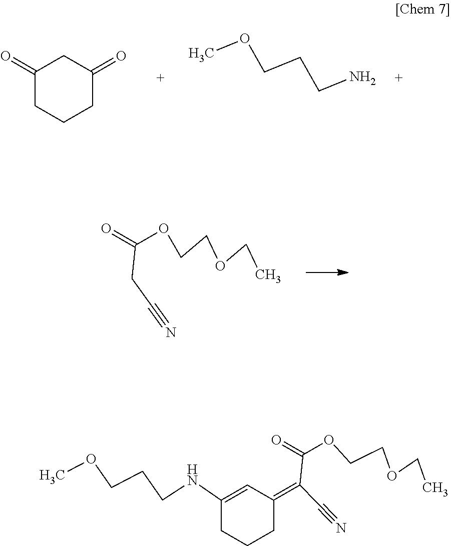

The synthesis illustrated by this scheme is particularly detailed in example A4 below.

### Hydrotrope

As mentioned previously, a composition according to the invention comprises at least one hydrotrone chosen from nicotinamide, caffeine, salicylic acid salts, the sodium salt of pyroglutamic acid (sodium PCA), sodium 1,3-benzenedisulfonate, sodium benzoate, sodium 4-pyridinecarboxylate, sodium benzenesulfonate, sodium p-toluenesulfonate (NaPTS), sodium butyl monoglycol sulfate (NaBMGS), 4-aminobenzoic acid HCl, sodium cumene sulfonate, N,N-diethyl nicotinamide, N-picolyl nicotinamide, N-allyl nicotinamide, 2-methacryloyloxyethyl phosphorylcholine, resorcinol, pyrogallol, N-picolylacetamide, procaine HCl, proline HCl, pyridine, 3-picolylamine, sodium ibuprofen, sodium xylene sulfonate (SXS), ethyl carbamate, pyridoxal hydrochloride, sodium benzoate, N,N-dimethylacetamide, N-methylacetamide, isoniazid, and mixtures thereof.

According to a preferred embodiment, a composition according to the invention comprises at least one hydrotrope chosen from nicotinamide, caffeine and mixtures thereof. In particular, such a composition may further comprise at least one additional hydrotrope chosen from salicylic acid salts, the sodium salt of pyroglutamic acid (sodium PCA), sodium 1,3-benzenedisulfonate, sodium benzoate, sodium 4-pyridinecarboxylate, sodium benzenesulfonate, sodium p-toluenesulfonate (NaPTS), sodium butyl monoglycol sulfate (NaBMGS), 4-aminobenzoic acid HCl, sodium cumene sulfonate, N,N-diethyl nicotinamide, N-picolyl nicotinamide, N-allyl nicotinamide, 2-methacryloyloxyethyl phosphorylcholine, resorcinol, pyrogallol, N-picolylacetamide, procaine HCl, proline HCl, pyridine, 3-picolylamine, sodium ibuprofen, sodium xylene sulfonate (SXS), ethyl carbamate, pyridoxal hydrochloride, sodium benzoate, N,N-dimethylacetamide, N-methylacetamide, isoniazid, and mixtures thereof, and preferably at least one salicylic acid salt.

According to a variant embodiment, a composition according to the invention comprises at least one hydrotrope chosen from nicotinamide, caffeine and mixtures thereof combined with at least one salicylic acid salt.

The salicylic acid salts may in particular be chosen from sodium salicylate, lysine salicylate, arginine salicylate, magnesium salicylate, and mixtures thereof.

Preferably, the salicylic acid salt is sodium salicylate.

According to a preferred embodiment, a composition according to the invention comprises at least nicotinamide as hydrotrope.

According to one particular embodiment, a composition according to the invention comprises nicotinamide and sodium salicylate.

The hydrotrope(s) are present in the composition according to the invention in an effective amount for solubilizing in water the merocyanine(s) of formula (I) as described above.

The content of hydrotrope(s) according to the invention present in the compositions according to the invention may range from 0.1% to 20% by weight, in particular from 0.1% to 10% by weight, preferably from 0.5% to 10% by weight, in particular from 0.5% to 3% by weight relative to the total weight of the composition.

In particular, the merocyanine(s) of formula (I))/hydrotrope(s) according to the invention weight ratio, of a composition according to the invention ranges from 0.01 to 8, preferably from 0.02 to 5, in particular from 0.05 to 3, and more particularly from 0.1 to 1.5.

According to one particular embodiment, a composition according to the invention comprises at least two different hydrotropes chosen from nicotinamide, caffeine, and sodium salicylate, and the merocyanine(s) of formula (I))/hydrotrope(s) weight ratio of a composition according to the invention ranges from 0.01 to 5, preferably from 0.02 to 3, in particular from 0.05 to 2, and more preferentially from 0.1 to 1.5.

According to one particular embodiment, a composition according to the invention comprises two different hydrotropes, preferably nicotinamide and caffeine, and the weight ratio between these two hydrotropes ranges from 0.2 to 5, in particular from 0.5 to 3, and more preferentially from 0.8 to 1.2.

### Aqueous Phase

A composition according to the invention comprises an aqueous phase and optionally an oily phase.

The aqueous phase comprises water and optionally a water-soluble organic solvent and is preferably single-phase.

According to a preferred embodiment, the composition according to the present invention has a water content of between 10% and 98% by weight and preferably between 25% and 90% by weight, and more preferentially between 35% and 85%, relative to the total weight of the composition.

According to the present invention, the term “water-soluble organic solvent” denotes a compound that is liquid at room temperature and water-miscible (miscibility with water of greater than 50% by weight at 25° C. and atmospheric pressure).

The water-soluble solvents that can be used in the composition of the invention may also be volatile. In particular, in order to obtain compositions having a BC3 content >0.9% use will be made of a water-soluble cosolvent in addition to the hydrotropes.

Among the water-soluble solvents that can be used in the composition according to the invention, mention may be made very particularly of lower monoalcohols containing from 1 to 5 carbon atoms, such as ethanol and isopropanol, polyols and also alkylene carbonates.

According to one preferred embodiment, the composition according to the invention also comprises at least one alcohol, in particular a monoalcohol, and preferably ethanol.

Preferably, in a composition according to the invention, the alcohol may be present in a content ranging from 0.5% to 30% by weight, better still from 2.0% to 25% by weight, preferably from 3.0% to 15% by weight, relative to the total weight of said composition.

According to a variant embodiment, the aqueous phase of a composition according to the invention may comprise at least one C₂-C₃₂ polyol.

Within the meaning of the present invention, the term “polyol” should be understood as meaning any organic molecule comprising at least two free hydroxyl groups.

Preferably, a polyol in accordance with the present invention is present in liquid form at room temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are suitable for formulating a composition according to the present invention are in particular those notably containing from 2 to 32 carbon atoms, preferably from 3 to 16 carbon atoms.

The polyol may be chosen, for example, from ethylene glycol, pentaerythritol, trimethylolpropane, propylene glycol, 1,3-propanediol, butylene glycol, isoprene glycol, pentylene glycol, hexylene glycol, C₃ and C₄ ketones and C₂-C₄ aldehydes, caprylyl glycol, glycerol, polyglycerols, such as glycerol oligomers, for instance diglycerol, polyethylene glycols, and mixtures thereof.

According to a preferred embodiment of the invention, said polyol is chosen from ethylene glycol, pentaerythritol, trimethylolpropane, pentylene glycol, propylene glycol, dipropylene glycol (DPG), caprylyl glycol, glycerol, polyglycerols, polyethylene glycols (PEG), and mixtures thereof.

According to one particular embodiment, the composition of the invention may comprise at least one polyol, in particular chosen from glycerol, caprylyl glycol, propylene glycol, pentylene glycol, dipropylene glycol (DPG), PEG-8, and mixtures thereof.

According to a preferred embodiment, the composition of the invention may comprise at least one polyol, in particular chosen from propylene glycol, pentylene glycol, dipropylene glycol (DPG), and mixtures thereof.

Preferably, the composition of the invention may comprise at least dipropylene glycol (DPG), as polyol.

When they are present, the polyol(s) is (are) preferably present in a composition according to the invention in a content ranging from 0.5% to 40% by weight, better still from 3% to 30% by weight, preferably from 5% to 25% by weight, relative to the total weight of said composition.

According to another particular embodiment, the composition of the invention may comprise at least one alkylene carbonate, in particular chosen from those of formula (II) below:

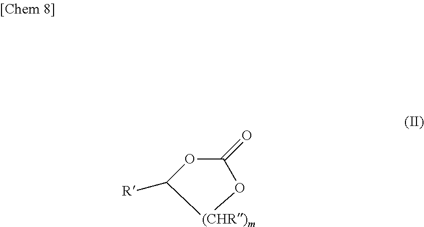

in which: R′ denotes a hydrogen atom, a linear or branched C₁-C₆ alkyl radical or a linear or branched C₁-C₄ hydroxyalkyl radical;
R″ represents a hydrogen atom, a linear or branched C₁-C₆ alkyl radical or a linear or branched C₁-C₄ hydroxyalkyl radical; and
m is 1, 2 or 3.

Preferably, the R′ radical represents a hydrogen atom, a linear or branched C₁-C₄ alkyl radical or a linear or branched C₁-C₂ hydroxyalkyl radical.

R″ represents a hydrogen atom, a linear or branched C₁-C₂ alkyl radical or a linear or branched C₁-C₂ hydroxyalkyl radical.

Preferably, m is 1.

As particularly advantageous examples of alkylene carbonates, mention may be made of the compounds for which the R′ radical represents a hydrogen atom (corresponding to ethylene carbonate), a methyl group (corresponding to propylene carbonate), an ethyl group (corresponding to 1,2-butylene carbonate), a hydroxymethyl group (R′═—CH₂OH; corresponding to glycerol carbonate).

Preferably, the alkylene carbonate used is propylene carbonate.

The alkyl or alkylene carbonates may be present in the compositions according to the invention at concentrations ranging preferably from 0.1% to 98% by weight, particularly from 0.5% to 50% by weight, more preferentially from 1% to 20% by weight and even more particularly from 1% to 10% by weight, notably 1% to 6% by weight, relative to the total weight of the composition.

As it emerges from the examples that appear below, an increase in the solubility of the merocyanines of formula (I) may be observed when one of the abovementioned organic solvents is used together with at least one hydrotrope. This effect is particularly advantageous for solubilizing, in an aqueous medium, a significant amount of merocyanine.

Thus, according to one variant embodiment, a composition according to the invention may further contain at least one hydrotrope, at least one organic solvent notably chosen from alcohols, in particular polyols and alkylene carbonates.

Besides the abovementioned solvents, a composition according to the invention may also comprise at least one organic solvent chosen from:
ketones that are liquid at room temperature, such as methyl ethyl ketone, methyl isobutyl ketone, diisobutyl ketone, isophorone, cyclohexanone or acetone;
cyclic ethers, such as γ-butyrolactone;
short-chain esters (having from 3 to 8 carbon atoms in total), such as ethyl acetate, butyl acetate, methyl acetate, propyl acetate, isopropyl acetate, isopentyl acetate, methoxypropyl acetate or butyl lactate;
ethers that are liquid at room temperature, such as diethyl ether, dimethyl ether or dichlorodiethyl ether;
alkanes that are liquid at room temperature, such as decane, heptane, dodecane or cyclohexane;
alkyl sulfoxides, such as dimethyl sulfoxide;
aldehydes that are liquid at room temperature, such as benzaldehyde or acetaldehyde;
3-ethyl ethoxypropionate;
acetals, such as methylal; and
mixtures thereof.

The aqueous phase may also comprise any water-soluble or water-dispersible compound that is compatible with an aqueous phase, such as stabilizers, gelling agents, film-forming polymers, thickeners, surfactants, and mixtures thereof.

Preferably, the aqueous phase is present in a composition according to the invention in a content ranging from 30% to 100% by weight, preferably from 35% to 99.5% by weight, and more preferentially from 45% to 99% by weight, relative to the total weight of said composition.

### Fatty Phase

As mentioned above, a composition according to the invention may comprise at least one fatty phase.

For the purposes of the invention, the term “fatty phase” means a phase comprising at least one oil and all of the liposoluble and lipophilic ingredients and the fatty substances used for the formulation of the compositions of the invention.

In particular, a composition according to the invention may comprise ranging from 0.5% to 80% by weight, preferably from 1% to 40% by weight and more preferentially from 3% to 20% by weight of fatty phase, relative to the total weight of the composition.

The fatty phase of the composition according to the invention may comprise oils, waxes, pasty compounds, and/or silicone compounds, and preferably at least one oil, notably a cosmetic oil.

### Oils

The term “oil” means a water-immiscible non-aqueous compound that is liquid at room temperature (20° C.) and atmospheric pressure (760 mmHg).

A fatty phase that is suitable for preparing the compositions, notably cosmetic compositions, according to the invention may comprise hydrocarbon oils, silicone oils, fluoro oils or non-fluoro oils, or mixtures thereof.

The oils may be volatile or non-volatile.

They may be of animal, plant, mineral or synthetic origin.

The fatty phase can comprise, besides the merocyanine screening agent(s) and optionally the lipophilic additional screening agent(s), at least one volatile or non-volatile hydrocarbon oil and/or one volatile and/or non-volatile silicone oil and/or one volatile and/or non-volatile fluoro oil.

For the purposes of the present invention, the term “hydrocarbon oil” is intended to mean an oil mainly containing hydrogen and carbon atoms.

The term “silicone oil” is intended to mean an oil comprising at least one silicon atom and notably at least one Si—O group.

The term “fluoro oil” means an oil comprising at least one fluorine atom.

The oils may optionally comprise oxygen, nitrogen, sulfur and/or phosphorus atoms, for example in the form of hydroxyl or acid radicals.

### Volatile Oils

For the purposes of the invention, the term “volatile oil” means any oil that is capable of evaporating on contact with the skin in less than one hour, at room temperature and atmospheric pressure. The volatile oil is a volatile cosmetic compound, which is liquid at room temperature, having in particular a non-zero vapour pressure, at room temperature and atmospheric pressure, in particular having a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The volatile oils may be hydrocarbon oils or silicone oils.

Among the volatile hydrocarbon oils containing from 8 to 16 carbon atoms, mention may be made notably of branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof.

Mention may also be made of volatile linear alkanes comprising from 8 to 16 carbon atoms, in particular from 10 to 15 carbon atoms, and more particularly from 11 to 13 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, mixtures of n-undecane (C₁₁) and of n-tridecane (C₁₃) obtained in Examples 1 and 2 of patent application WO 2008/155059 from the company Cognis, and mixtures thereof.

Volatile silicone oils that may be mentioned include volatile linear silicone oils such as hexamethyldisiloxane, octamethyltrisiloxane, decamethyltetrasiloxane, tetradecamethylhexasiloxane, hexadecamethylheptasiloxane and dodecamethylpentasiloxane.

Volatile cyclic silicone oils that may be mentioned include hexamethylcyclotrisiloxane, octamethylcyclotetrasiloxane, decamethylcyclopentasiloxane, cyclohexasiloxane and dodecamethylcyclohexasiloxane, and in particular cyclohexasiloxane.

Use may also be made of volatile fluoro oils, such as nonafluoromethoxybutane, decafluoropentane, tetradecafluorohexane, dodecafluoropentane, and mixtures thereof.

Non-volatile oils

The term “non-volatile” is intended to mean to an oil of which the vapour pressure at room temperature and atmospheric pressure is non-zero and is less than 10⁻³ mmHg (0.13 Pa).

The non-volatile oils may notably be chosen from non-volatile hydrocarbon, fluoro and/or silicone oils.

Non-volatile hydrocarbon oils that may notably be mentioned include:
hydrocarbon oils of animal origin,
linear or branched hydrocarbons of mineral or synthetic origin, such as petroleum jelly, polydecenes, hydrogenated polyisobutene such as Parleam, squalane, and mixtures thereof,
non-volatile alkanes, preferably with a viscosity of less than 20 mPa·s at 20° C. measured with a Rheomat RM100® viscometer from Lamy Rheology. The term “non-volatile alkane” means a hydrocarbon cosmetic oil which is liquid at room temperature, notably having a vapour pressure at 20° C. of less than 0.01 kPa, according to the definition of a Volatile Organic Compound (VOC) of article 2 of European Council Directive 1999/13/EC of 11 Mar. 1992: “Any organic compound having, at a temperature of 293.15 K, a vapour pressure of 0.01 kPa or more”. In particular, the non-volatile alkanes comprise from 10 to 30 carbon atoms, in particular from 12 to 26 carbon atoms, and more particularly from 15 to 19 carbon atoms, and preferably a mixture of alkanes containing from 15 to 19 carbon atoms, for example the products sold under the references Emogreen L19 and Emosmart L19 from SEPPIC,
hydrocarbon oils of plant origin, such as glyceride triesters, which are generally triesters of fatty acids and of glycerol, the fatty acids of which may have varied chain lengths from 4 to 24 carbon atoms, it being possible for these chains to be saturated or unsaturated and linear or branched; these oils are notably wheatgerm oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame oil, marrow oil, blackcurrant oil, evening primrose oil, millet oil, barley oil, quinoa oil, rye oil, safflower oil, candlenut oil, passionflower oil and musk rose oil; or else caprylic/capric acid triglycerides, such as those sold by the company Stéarinerie Dubois or those sold under the names Miglyol 810, 812 and 818 by the company Dynamit Nobel,
synthetic ethers containing from 10 to 40 carbon atoms, such as dicapryl ether,
synthetic esters, such as the oils of formula R₁COOR₂, in which R₁ represents a linear or branched fatty acid residue comprising from 1 to 40 carbon atoms and R₂ represents a hydrocarbon chain that is notably branched, containing from 1 to 40 carbon atoms, with the proviso that R₁+R₂ is greater than or equal to 10, for instance purcellin oil (cetostearyl octanoate), isopropyl myristate, isopropyl palmitate, alkyl benzoates having between 12 and 15 carbon atoms, such as the product sold under the trade name Finsolv TN or Witconol TN by the company Witco or Tegosoft TN by the company Evonik Goldschmidt, 2-ethylphenyl benzoate, such as the commercial product sold under the name X-Tend 226 by the company ISP, isopropyl lanolate, hexyl laurate, diisopropyl adipate, isononyl isononanoate, oleyl erucate, 2-ethylhexyl palmitate, isostearyl isostearate, diisopropyl sebacate such as the product sold under the name Dub Dis by the company Stearinerie Dubois, alcohol or polyalcohol octanoates, decanoates or ricinoleates, such as propylene glycol dioctanoate; hydroxylated esters, such as isostearyl lactate, diisostearyl malate; and pentaerythritol esters; citrates, such as the ester of C₃-C₂₂ tricarboxylic acid and of C₁-C₆ alcohols with the INCI name Triethyl Citrate, for example the one sold under the name Citrofol AI Extra by the company Jungbunzlauer; tartrates, such as linear dialkyl tartrates having 12 and 13 carbon atoms, for example those sold under the name Cosmacol ETI by the company Enichem Augusta Industriale, and also linear dialkyl tartrates having between 14 and 15 carbon atoms, for example those sold under the name Cosmacol ETL by the same company, and acetates,
fatty amides, such as isopropyl N-lauroyl sarcosinate, for example the product sold under the trade name Eldew SL205 from Ajinomoto,
polyol esters and pentaerythritol esters, for instance dipentaerythritol tetrahydroxystearate/tetraisostearate,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol,
C₁₂-C₂₂ higher fatty acids, such as oleic acid, linoleic acid, linolenic acid, and mixtures thereof,
carbonates, such as dicaprylyl carbonate, for example the product sold under the name Cetiol CC by the company Cognis;
non-phenyl silicone oils, for instance caprylyl methicone, and
phenyl silicone oils, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, trimethylpentaphenyltrisiloxane, and mixtures thereof;
and also mixtures of these various oils.

### Other Fatty Substances

A fatty phase according to the invention can additionally comprise other fatty substances, mixed with or dissolved in an oil.

The other fatty substances which can be present in the oily phase are, for example:
fatty acids comprising from 8 to 30 carbon atoms, such as stearic acid, lauric acid, palmitic acid and oleic acid;
waxes, other than glyceryl trihydroxystearate, such as lanolin, beeswax, carnauba or candelilla wax, paraffin waxes, lignite waxes or microcrystalline waxes, ceresin or ozokerite, or synthetic waxes, such as polyethylene waxes or Fischer-Tropsch waxes;
silicone resins such as trifluoromethyl(C₁-C₄)alkyldimethicone and trifluoropropyl dimethicone;
silicone elastomers such as the products sold under the name KSG by the company Shin-Etsu, under the names Trefil or BY29 by the company Dow Corning or under the name Gransil by the company Grant Industries;
a gum chosen from silicone gums (dimethiconol);
a pasty compound, such as polymeric or non-polymeric silicone compounds, esters of an oligomeric glycerol, arachidyl propionate, fatty acid triglycerides and derivatives thereof;
and mixtures thereof.

As fatty substances, a composition according to the invention may comprise at least one fatty alcohol wax. Such waxes may be chosen from lauric or lauryl alcohol, myristic or myristyl alcohol, cetyl alcohol, stearyl alcohol, arachidyl alcohol, behenyl alcohol, lignoceryl alcohol, ceryl alcohol, montanyl alcohol, myricyl alcohol, and mixtures thereof.

As fatty substance, a composition according to the invention may in particular comprise at least one butter, in particular a plant butter.

The plant butter(s) suitable for use in the invention is (are) preferably chosen from the group comprising avocado butter, cocoa butter, shea butter, kokum butter, mango butter, murumuru butter, coconut butter, apricot kernel butter, sal butter and urucum butter, and mixtures thereof, and in particular is shea butter.

These fatty substances may be chosen in a varied manner by a person skilled in the art in order to prepare a composition having the desired properties, for example in terms of consistency or texture.

According to a preferred embodiment, a composition according to the invention comprises at least one non-volatile oil, preferably at least one non-volatile hydrocarbon oil, in particular chosen from linear or branched hydrocarbon is of mineral or synthetic origin, non-volatile alkanes, hydrocarbon oils of plant origin, synthetic esters, fatty amides, carbonates, and also the mixtures of these various oils.

In particular, such non-volatile hydrocarbon oils may be present in a composition according to the invention in a content ranging from 2.0% to 20.0% by weight, preferably from 2.0% to 15.0% by weight, relative to the total weight of the composition.

According to a preferred embodiment, a composition according to the invention contains at least one oil chosen from squalane, mixtures of alkanes containing from 15 to 19 carbon atoms, caprylic/capric acid triglycerides, alkyl benzoates having between 12 and 15 carbon atoms, diisopropyl adipate, 2-ethylhexyl palmitate, diisopropyl sebacate, citrates, the ester of C₃-C₂₂ tricarboxylic acid and of C₁-C₆ alcohols with the INCI name Triethyl Citrate, isopropyl N-lauroyl sarcosinate, dicaprylyl carbonate, and mixtures thereof.

Preferably, a composition according to the invention comprises less than 2.0% by weight of silicone oil(s), in particular less than 1.0% by weight of silicone oil(s), preferably less than 0.5% by weight of silicone oil(s), relative to the total weight of the composition, and more preferentially is free of silicone oil(s).

Preferably, a composition according to the invention comprises a fatty phase containing at least one fatty substance.

According to a preferred embodiment, a composition according to the invention comprises at least one non-volatile hydrocarbon oil, and preferably at least one non-volatile ester oil.

According to a preferred embodiment, a composition according to the invention further comprises at least one solid fatty substance, in particular a fatty acid containing from 8 to 30 carbon atoms, and more preferentially stearic acid.

### Additional UV-Screening Agents

A composition according to the invention may comprise at least one additional UV-screening agent other than the merocyanines of formula (I) previously described.

In particular, a composition according to the invention may comprise at least one compound for screening out UVA and/or UVB other than the merocyanines of formula (I) previously described.

The compositions according to the invention may thus also contain one or more additional UV-screening agents chosen from hydrophilic, lipophilic or insoluble organic UV-screening agents and/or one or more mineral pigments. It will preferentially be constituted of at least one hydrophilic, lipophilic or insoluble organic UV-screening agent.

The term “hydrophilic UV-screening agent” is intended to mean any cosmetic or dermatological, organic or inorganic compound for screening out UV radiation, which can be fully dissolved in molecular form in a liquid aqueous phase or else which can be solubilized in colloidal form (for example in micellar form) in a liquid aqueous phase.

The term “lipophilic screening agent” is intended to mean any cosmetic or dermatological organic or inorganic compound for screening out UV radiation, which can be fully dissolved in molecular form in a liquid fatty phase or else which can be solubilized in colloidal form (for example in micellar form) in a liquid fatty phase.

The term “insoluble organic UV-screening agent” is intended to mean any cosmetic or dermatological organic or inorganic compound for screening out UV radiation, which has a solubility in water of less than 0.5% by weight and a solubility of less than 0.5% by weight in the majority of organic solvents such as liquid paraffin, fatty alcohol benzoates and fatty acid triglycerides, for example Miglyol 812® sold by the company Dynamit Nobel. This solubility, determined at 70° C., is defined as the amount of product in solution in the solvent at equilibrium with an excess of solid in suspension after returning to room temperature. It can be easily evaluated in the laboratory.

The additional organic UV-screening agents are chosen in particular from cinnamic compounds; anthranilate compounds; salicylic compounds; dibenzoylmethane compounds; benzylidenecamphor compounds; benzophenone compounds; β,β-diphenylacrylate compounds; triazine compounds; benzotriazole compounds; benzalmalonate compounds, in particular those cited in patent U.S. Pat. No. 5,624,663; benzimidazole derivatives; imidazoline compounds; bis-benzoazolyl compounds, such as described in patents EP 0 669 323 and U.S. Pat. No. 2,463,264; p-aminobenzoic (PABA) compounds; methylenebis(hydroxyphenylbenzotriazole) compounds, such as described in applications U.S. Pat. Nos. 5,237,071, 5,166,355, GB 2 303 549, DE 197 26 184 and EP 0 893 119; benzoxazole compounds, such as described in patent applications EP 0 832 642, EP 1 027 883, EP 1 300 137 and DE 101 62 844; screening polymers and screening silicones, such as those described in particular in application WO 93/04665; dimers derived from α-alkylstyrene, such as those described in patent application DE 198 55 649; 4,4-diarylbutadiene compounds, such as described in applications EP 0 967 200, DE 197 46 654, DE 197 55 649, EP 1 008 586, EP 1 133 980 and EP 0 133 981, and mixtures thereof.

Mention may be made, as examples of organic photoprotective agents, of those denoted below under their INCI names.

### Cinnamic Compounds:

Ethylhexyl Methoxycinnamate, sold in particular under the trade name Parsol MCX® by DSM Nutritional Products,
Isopropyl Methoxycinnamate,
Isoamyl p-Methoxycinnamate, sold under the trade name Neo Heliopan E 1000® by Symrise,
DEA Methoxycinnamate,
Diisopropyl Methylcinnamate,
Glyceryl Ethylhexanoate Dimethoxycinnamate.

### Dibenzoylmethane Compounds:

Butyl Methoxydibenzoylmethane, sold in particular under the trade name Parsol 1789® by DSM Nutritional Products,
Isopropyl Dibenzoylmethane.

### Para-Aminobenzoic Compounds:

PABA,
Ethyl PABA,
Ethyl Dihydroxypropyl PABA,
Ethylhexyl Dimethyl PABA, sold in particular under the name Escalol 507® by ISP,
Glyceryl PABA,
PEG-25 PABA, sold under the name Uvinul P 25® by BASF.

### Salicylic Compounds:

Homosalate, sold under the name Eusolex HMS® by Rona/EM Industries,
Ethylhexyl Salicylate, sold under the name Neo Heliopan OS® by Symrise,
Dipropylene Glycol Salicylate, sold under the name Dipsal® by Scher,
TEA Salicylate, sold under the name Neo Heliopan TS® by Symrise,

### (β,β-Diphenylacrylate Compounds:

Octocrylene, sold in particular under the trade name Uvinul N 539® by BASF,
Etocrylene, sold in particular under the trade name Uvinul N 35® by BASF.

### Benzophenone Compounds:

Benzophenone-1, sold under the trade name Uvinul 400® by BASF,
Benzophenone-2, sold under the trade name Uvinul D 50® by BASF,
Benzophenone-3 or Oxybenzone, sold under the trade name Uvinul M 40® by BASF,
Benzophenone-4, sold under the trade name Uvinul MS 40® by BASF,
Benzophenone-5,
Benzophenone-6, sold under the trade name Helisorb 11® by Norquay,
Benzophenone-8, sold under the trade name Spectra-Sorb UV-24® by American Cyanamid,
Benzophenone-9, sold under the trade name Uvinul DS 49® by BASF,
Benzophenone-12,
n-Hexyl 2-(4-diethylamino-2-hydroxybenzoyl)benzoate, sold under the trade name Uvinul A Plus® or, as a mixture with octyl methoxycinnamate, under the trade name Uvinul A Plus B® by BASF,
1,1′-(1,4-Piperazinediyl)bis[1-[2-[4-(diethylamino)-2-hydroxybenzoyl]phenyl]methanone](CAS 919803-06-8), as described in application WO 2007/071584; this compound advantageously being used in micronized form (mean size of 0.02 to 2 μm), which may be obtained, for example, according to the micronization process described in applications GB 2 303 549 and EP 0 893 119, and in particular in the form of an aqueous dispersion.

### Benzylidene Camphor Compounds:

3-Benzylidene Camphor, manufactured under the name Mexoryl SD® by Chimex,
4-Methylbenzylidene Camphor, sold under the name Eusolex 6300® by Merck,
Benzylidene Camphor Sulfonic Acid, manufactured under the name Mexoryl SL® by Chimex,
Camphor Benzalkonium Methosulfate, manufactured under the name Mexoryl SO® by Chimex,
Terephthalylidene Dicamphor Sulfonic Acid, manufactured under the name Mexoryl SX® by Chimex,
Polyacrylamidomethyl Benzylidene Camphor, manufactured under the name Mexoryl SW® by Chimex,

### Phenylbenzimidazole Compounds:

Phenylbenzimidazole Sulfonic Acid, sold in particular under the trade name Eusolex 232® by Merck.

### Bis-Benzoazolyl Compounds:

Disodium Phenyl Dibenzimidazole Tetrasulfonate, sold under the trade name Neo Heliopan AP® by Haarmann and Reimer.

### Phenylbenzotriazole Compounds:

Drometrizole Trisiloxane, sold under the name Silatrizole® by Rhodia Chimie.

### Methylenebis(Hydroxyphenylbenzotriazole) Compounds:

Methylene bis-Benzotriazolyl Tetramethylbutylphenol, in particular in solid form, such as the product sold under the trade name Mixxim BB/100® by Fairmount Chemical, or in the form of an aqueous dispersion of micronized particles with a mean particle size ranging from 0.01 to 5 μm, more preferentially from 0.01 to 2 μm and more particularly from 0.020 to 2 μm, with at least one alkyl polyglycoside surfactant having the structure CₙH₂ₙ₊₁O(C₆H₁₀O₅)ₓH, in which n is an integer from 8 to 16 and x is the mean degree of polymerization of the (C₆H₁₀O₅) unit and ranges from 1.4 to 1.6, as described in patent GB 2 303 549, sold in particular under the trade name Tinosorb M® by BASF, or in the form of an aqueous dispersion of micronized particles with a mean particle size ranging from 0.02 to 2 μm, more preferentially from 0.01 to 1.5 μm and more particularly from 0.02 to 1 μm, in the presence of at least one polyglyceryl mono(C₈-C₂₀)alkyl ester with a degree of glycerol polymerization of at least 5, such as the aqueous dispersions described in application WO 2009/063392.

### Triazine Compounds:

3,3′-(1,4-Phenylene)bis(5,6-diphenyl-1,2,4-triazine), with the INCI name Phenylene Bis-Diphenyltriazine, and with the following chemical structure:

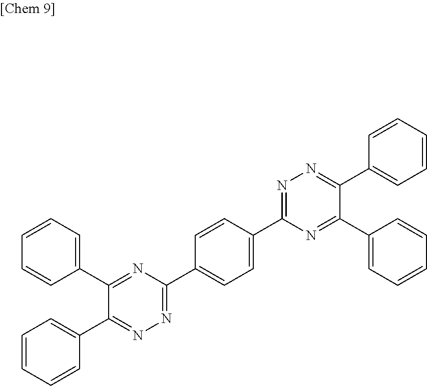 [SMILES: c1ccc(-c2nnc(-c3ccc(-c4nnc(-c5ccccc5)c(-c5ccccc5)n4)cc3)nc2-c2ccccc2)cc1]

Bis-Ethylhexyloxyphenol Methoxyphenyl Triazine, sold under the trade name Tinosorb S® by BASF,
Ethylhexyl Triazone, sold in particular under the trade name Uvinul T 150® by BASF,
Diethylhexyl Butamido Triazone, sold under the trade name Uvasorb HEB® by Sigma 3V,
2,4,6-tris(dineopentyl 4′-aminobenzalmalonate)-s-triazine,
2,4,6-tris(diisobutyl 4′-aminobenzalmalonate)-s-triazine,
2,4-bis(n-butyl 4′-aminobenzoate)-6-(aminopropyltrisiloxane)-s-triazine,
2,4-bis(dineopentyl 4′-aminobenzalmalonate)-6-(n-butyl 4′-aminobenzoate)-s-triazine,
symmetrical triazine screening agents substituted by naphthalenyl groups or polyphenyl groups described in patent U.S. Pat. No. 6,225,467, application WO 2004/085412 (see compounds 6 and 9) or the document “Symmetrical Triazine Derivatives”, IP.COM IPCOM000031257 Journal, INC West Henrietta, NY, US (20 Sep. 2004), in particular 2,4,6-tris(diphenyl)triazine and 2,4,6-tris(terphenyl)triazine, which is also mentioned in patent applications WO 06/035000, WO 06/034982, WO 06/034991, WO 06/035007, WO 2006/034992 and WO 2006/034985, these compounds advantageously being used in micronized form (mean particle size of 0.02 to 3 μm), which can be obtained, for example, according to the micronization process described in applications GB-A-2 303 549 and EP-A-893 119, and in particular in aqueous dispersion form,
silicone triazines substituted with two aminobenzoate groups, as described in patent EP 0 841 341, in particular 2,4-bis(n-butyl 4′-aminobenzalmalonate)-6-[(3-{1,3,3,3-tetramethyl-1-[(trimethylsilyl)oxy]disiloxanyl}propyl)amino]-s-triazine.

### Anthranilic Compounds:

Menthyl Anthranilate, sold under the trade name Neo Heliopan MA® by Symrise.

### Imidazoline Compounds:

Ethylhexyl Dimethoxybenzylidene Dioxoimidazoline Propionate.

### Benzalmalonate Compounds:

Polyorganosiloxane comprising benzalmalonate functions, such as Polysilicone-15, sold under the trade name Parsol SLX® by Hoffmann-LaRoche.

### 4,4-Diarylbutadiene Compounds:

1,1-Dicarboxy(2,2′-dimethylpropyl)-4,4-diphenylbutadiene.

### Benzoxazole Compounds:

2,4-bis[5-1(dimethylpropyl)benzoxazol-2-yl-(4-phenyl)imino]-6-(2-ethylhexyl)imino-1,3,5-triazine sold under the name Uvasorb K2A® by Sigma 3V.

The preferential organic screening agents are chosen from:
Ethylhexyl Methoxycinnamate,
Ethylhexyl Salicylate,
Homosalate,
Butyl Methoxydibenzoylmethane,
Octocrylene,
Phenylbenzimidazole Sulfonic Acid,
Benzophenone-3,
Benzophenone-4,
Benzophenone-5,
n-Hexyl 2-(4-diethylamino-2-hydroxybenzoyl)benzoate,
4-Methylbenzylidene Camphor,
Terephthalylidene Dicamphor Sulfonic Acid,
Disodium Phenyl Dibenzimidazole Tetrasulfonate,
Methylene bis-Benzotriazolyl Tetramethylbutylphenol,
Bis-Ethylhexyloxyphenol Methoxyphenyl Triazine,
Ethylhexyl Triazone,
Diethylhexyl Butamido Triazone,
2,4,6-Tris(dineopentyl 4′-aminobenzalmalonate)-s-triazine,
2,4,6-Tris(diisobutyl 4′-aminobenzalmalonate)-s-triazine,
2,4-Bis(n-butyl 4′-aminobenzoate)-6-(aminopropyltrisiloxane)-s-triazine,
2,4-Bis(dineopentyl 4′-aminobenzalmalonate)-6-(n-butyl 4′-aminobenzoate)-s-triazine,
2,4-Bis(n-butyl 4′-aminobenzalmalonate)-6-[(3-{1,3,3,3-tetramethyl-1-[(trimethylsilyloxy)]disiloxanyl}propyl)amino]-s-triazine,
2,4,6-Tris(diphenyl)triazine,
2,4,6-Tris(terphenyl)triazine,
Drometrizole Trisiloxane,
Polysilicone-15,
1,1-Dicarboxy(2,2′-dimethylpropyl)-4,4-diphenylbutadiene,
2,4-Bis-[5-1(dimethylpropyl)benzoxazol-2-yl-(4-phenyl)-imino]-6-(2-ethylhexyl)imino-1,3,5-triazine,
and mixtures thereof.

The particularly preferred organic screening agents are chosen from:
Ethylhexyl salicylate,
Homosalate,
Butyl Methoxydibenzoylmethane,
Octocrylene,
n-Hexyl 2-(4-diethylamino-2-hydroxybenzoyl)benzoate,
Terephthalylidene Dicamphor Sulfonic Acid,
Bis-Ethylhexyloxyphenol Methoxyphenyl Triazine,
Ethylhexyl Triazone,
Diethylhexyl Butamido Triazone,
2,4-Bis(n-butyl 4′-aminobenzalmalonate)-6-[(3-{1,3,3,3-tetramethyl-1-[(trimethylsilyloxy)]disiloxanyl}propyl)amino]-s-triazine,
Drometrizole Trisiloxane,
and mixtures thereof.

The inorganic UV-screening agents used in accordance with the present invention are metal oxide pigments. More preferentially, the inorganic UV-screening agents of the invention are metal oxide particles having a mean elementary particle size of less than or equal to 0.5 μm, more preferentially of between 0.005 and 0.5 μm, more preferentially still of between 0.01 and 0.2 μm, better still between 0.01 and 0.1 μm and more particularly between 0.015 and 0.05 μm.

They may be chosen in particular from titanium oxide, zinc oxide, iron oxide, zirconium oxide and cerium oxide, or mixtures thereof.

Such coated or uncoated metal oxide pigments are described in particular in patent application EP 0 518 773. As commercial pigments, mention may be made of the products sold by the companies Sachtleben Pigments, Tayca, Merck and Degussa.

The metal oxide pigments can be coated or uncoated.

The coated pigments are pigments which have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminium salts of fatty acids, metal alkoxides (of titanium or aluminium), polyethylene, silicones, proteins (collagen, elastin), alkanolamines, silicon oxides, metal oxides or sodium hexametaphosphate.

The coated pigments are more particularly titanium oxides coated:
with silica, such as the product Sunveil® from the company Ikeda,
with silica and iron oxide, such as the product Sunveil F® from the company Ikeda,
with silica and alumina, such as the products Microtitanium Dioxide MT 500 SA® and Microtitanium Dioxide MT 100 SA® from the company Tayca and Tioveil from the company Tioxide,
with alumina, such as the products Tipaque TTO-55 (B)® and Tipaque TTO-55 (A)® from the company Ishihara and UVT 14/4 from the company Sachtleben Pigments,
with alumina and aluminium stearate, such as the products Microtitanium Dioxide MT 100 T®, MT 100 TX®, MT 100 Z® and MT-01® from the company Tayca, the products Solaveil CT-10 W® and Solaveil CT 100® from the company Uniqema and the product Eusolex T-AVO® from the company Merck,
with silica, alumina and alginic acid, such as the product MT-100 AQ® from the company Tayca,
with alumina and aluminium laurate, such as the product Microtitanium Dioxide MT 100 S® from the company Tayca,
with iron oxide and iron stearate, such as the product Microtitanium Dioxide MT 100 F® from the company Tayca,
with zinc oxide and zinc stearate, such as the product BR 351® from the company Tayca,
with silica and alumina and treated with a silicone, in particular the products Microtitanium Dioxide MT 600 SAS®, Microtitanium Dioxide MT 500 SAS® or Microtitanium Dioxide MT 100 SAS® from the company Tayca,
with silica, alumina and aluminium stearate and treated with a silicone, such as the product STT-30-DS® from the company Titan Kogyo,
with silica and treated with a silicone, such as the product UV-Titan X 195® from the company Sachtleben Pigments,
with alumina and treated with a silicone, such as the products Tipaque TTO-55 (S)® from the company Ishihara or UV Titan M 262® from the company Sachtleben Pigments,
with triethanolamine, such as the product STT-65-S from the company Titan Kogyo,
with stearic acid, such as the product Tipaque TTO-55 (C)® from the company Ishihara,
with sodium hexametaphosphate, such as the product Microtitanium Dioxide MT 150 W® from the company Tayca,
TiO₂ treated with octyltrimethylsilane, sold under the trade name T 805® by the company Degussa Silices,
TiO₂ treated with a polydimethylsiloxane, sold under the trade name 70250 Cardre UF TiO₂SI₃® by the company Cardre,
anatase/rutile TiO₂ treated with a polydimethylhydrogenosiloxane, sold under the trade name Microtitanium Dioxide USP Grade Hydrophobic® by the company Color Techniques.
TiO₂ coated with triethylhexanoin, with aluminium stearate and with alumina sold under the trade name Solaveil CT-200-LQ-(WD) by Croda,
TiO₂ coated with aluminium stearate, with alumina and with silicone sold under the trade name Solaveil CT-12W-LQ-(WD) by Croda,
TiO₂ coated with lauroyl lysine sold by Daito Kasei Kogyo under the name LL 5 Titanium Dioxide CR 50,
TiO₂ coated with C₉-C₁₅ fluoroalcohol phosphate and with aluminium hydroxide sold by Daito Kasei Kogyo under the name PFX-5 TiO₂ CR-50.

Mention may also be made of TiO₂ pigments doped with at least one transition metal, such as iron, zinc or manganese and more particularly manganese. Preferably, said doped pigments are in the form of an oily dispersion. The oil present in the oily dispersion is preferably chosen from triglycerides, including those of capric/caprylic acids. The oily dispersion of titanium oxide particles may further comprise one or more dispersants, for instance a sorbitan ester, such as sorbitan isostearate, or a polyoxyalkylenated fatty acid ester of glycerol, such as TRI-PPG-3 myristyl ether citrate and polyglyceryl-3 polyricinoleate.

Preferably, the oily dispersion of titanium oxide particles comprises at least one dispersant chosen from polyoxyalkylenated fatty acid esters of glycerol. Mention may be made more particularly of the oily dispersion of TiO₂ particles doped with manganese in capric/caprylic acid triglyceride in the presence of TRI-PPG-3 myristyl ether citrate and polyglyceryl-3 polyricinoleate and sorbitan isostearate having the INCI name: Titanium Dioxide (and) Tri-PPG-3 Myristyl Ether Citrate (and) Polyglyceryl-3 Polyricinoleate (and) Sorbitan Isostearate, such as the product sold under the trade name Optisol TD50® by the company Croda.

The uncoated titanium oxide pigments are sold, for example, by the company Tayca under the trade names Microtitanium Dioxide MT 500 B or Microtitanium Dioxide MT 600 B®, by the company Degussa under the name P 25, by the company Wacker under the name Transparent titanium oxide PW®, by the company Miyoshi Kasei under the name UFTR®, by the company Tomen under the name ITS® and by the company Tioxide under the name Tioveil AQ.

The uncoated zinc oxide pigments are, for example:
those sold under the name Z-Cote by the company Sunsmart;
those sold under the name Nanox® by the company Elementis;
those sold under the name Nanogard WCD 2025® by the company Nanophase Technologies.

The coated zinc oxide pigments are, for example:
those sold under the name Oxide Zinc CS-5® by the company Toshibi (ZnO coated with polymethylhydrogenesiloxane);
those sold under the name Nanogard Zinc Oxide FN® by the company Nanophase Technologies (as a 40% dispersion in Finsolv TN®, benzoate of C₁₂-C₁₅ alcohols),
those sold under the name Daitopersion Zn-30® and Daitopersion Zn-50® by the company Daito (dispersions in cyclopolymethylsiloxane/oxyethylenated polydimethylsiloxane, containing 30% or 50% of zinc oxides coated with silica and polymethylhydrosiloxane);
those sold under the name NFD Ultrafine ZnO® by the company Daikin (ZnO coated with perfluoroalkyl phosphate and copolymer based on perfluoroalkylethyl as a dispersion in cyclopentasiloxane);
those sold under the name SPD-Z1® by the company Shin-Etsu (ZnO coated with silicone-grafted acrylic polymer, dispersed in cyclodimethylsiloxane);
those sold under the name Escalol Z100® by the company ISP (alumina-treated ZnO dispersed in an ethylhexyl methoxycinnamate/PVP-hexadecene copolymer/methicone mixture);
those sold under the name Fuji ZnO-SMS-10® by the company Fuji Pigment (ZnO coated with silica and polymethylsilsesquioxane);
those sold under the name Nanox Gel TN® by the company Elementis (ZnO dispersed at a concentration of 55% in benzoate of C₁₂-C₁₅ alcohols with hydroxystearic acid polycondensate).

The uncoated cerium oxide pigments may, for example, be those sold under the name Colloidal Cerium Oxide® by the company Rhone-Poulenc.

The uncoated iron oxide pigments are sold, for example, by the company Arnaud under the names Nanogard WCD 2002® (FE 45B®), Nanogard Iron FE 45 BL AQ, Nanogard FE 45R AQ® and Nanogard WCD 2006® (FE 45R®) or by the company Mitsubishi under the name TY-220®.

The coated iron oxide pigments are sold, for example, by the company Arnaud under the names Nanogard WCD 2008 (FE 45B FN®), Nanogard WCD 2009® (FE 45B 556®), Nanogard FE 45 BL 345® and Nanogard FE 45 BL® or by the company BASF under the name Transparent Iron Oxide®.

Mention may also be made of mixtures of metal oxides, especially of titanium dioxide and of cerium dioxide, including the equal-weight mixture of titanium dioxide and cerium dioxide coated with silica, sold by the company Ikeda under the name Sunveil A®, and also the mixture of titanium dioxide and zinc dioxide coated with alumina, silica and silicone, such as the product M 261® sold by the company Sachtleben Pigments, or coated with alumina, silica and glycerol, such as the product M 211® sold by the company Sachtleben Pigments.

According to the invention, coated or uncoated titanium oxide pigments are particularly preferred.

According to one preferred embodiment, the composition comprises one or more lipophilic UV-screening agents.

According to one preferred embodiment, the composition comprises one or more UV-screening agents chosen from lipophilic UVA-screening agents and/or lipophilic UVB-screening agents.

More preferentially, the composition comprises one or more UV-screening agents chosen from dibenzoylmethane derivatives, salicylic derivatives, β,β-diphenyl acrylate derivatives, triazine derivatives and benzylidene camphor derivatives.

According to one particularly preferred embodiment, the composition comprises one or more UV-screening agents chosen from Terephthalylidene Dicamphor Sulfonic Acid, Butyl Methoxydibenzoylmethane, Homosalate, Ethylhexyl Salicylate, Octocrylene, Ethylhexyl Triazone, and mixtures thereof.

According to one embodiment, the amount of the organic UV-screening agent(s) present in the composition according to the invention may range from 0.5% to 50% by weight, relative to the total weight of the composition. It ranges for example from 1% to 40% by weight, or else for example from 5% to 35% by weight, and even for example ranges from 10% to 35% by weight, relative to the total weight of the composition.

According to one particular embodiment, the concentration of UV-screening agents in the composition according to the invention ranges from 1% to 50% and preferably from 1% to 40% by weight, relative to the total weight of the composition.

According to one particular embodiment, the composition according to the invention comprises at least one mineral UV-screening agent.

According to one particular embodiment, the amount of the mineral UV-screening agent(s) present in the composition according to the invention may range from 0.01% to 20% by weight, relative to the total weight of the composition. It ranges, for example, from 1% to 15% by weight, relative to the total weight of the composition.

According to a particular embodiment, the composition according to the invention also comprises one or more organic UV-screening agents and one or more mineral UV-screening agents.

According to one particular embodiment, the composition according to the invention also comprises a combination of UV-screening agents as described in patent FR 2 977 490, application WO 2013/004777 or application US 2014/0134120.

### Surfactants

According to a preferred embodiment, the composition according to the invention may also comprise at least one surfactant.

The surfactants may be chosen from non-ionic, anionic, cationic and amphoteric surfactants, and mixtures thereof. Reference may be made to the Kirk-Othmer Encyclopedia of Chemical Technology, volume 22, pages 333-432, 3rd Edition, 1979, Wiley, for the definition of the emulsifying properties and functions of surfactants, in particular pages 347-377 of this reference, for anionic, amphoteric and non-ionic surfactants.

### Non-Ionic Surfactant

Preferably, the composition according to the invention comprises at least one non-ionic surfactant.

The non-ionic surfactants may in particular be chosen from alkyl and polyalkyl esters of poly(ethylene oxide), oxyalkylenated alcohols, alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, glyceryl esters, optionally polyoxyethylenated alkyl and polyalkyl esters of glycerol, optionally polyoxyethylenated alkyl and polyalkyl ethers of glycerol, gemini surfactants, cetyl alcohol, stearyl alcohol, and mixtures thereof.

As glyceryl esters, mention may notably be made of C₁₆-C₂₂ fatty acid esters of glycerol, in particular glyceryl esters of a fatty acid having 18 carbon atoms, and polyglyceryl esters of a fatty acid containing from 8 to 12 carbon atoms.

Glyceryl esters of a C₁₈ fatty acid that may notably be mentioned include glyceryl stearate (glyceryl monostearate, distearate and/or tristearate) (CTFA name: glyceryl stearate) or glyceryl ricinoleate, or mixtures thereof.

As fatty acid ester of glycerol, mention may be made of mixtures based on glyceryl stearate, such as the mixture of glyceryl stearate and polyethylene glycol 100 OE monostearate, and in particular the one comprising a 50/50 mixture, sold under the name Arlacel 165® by the company Croda, or else the product containing glyceryl stearate (glyceryl mono-distearate) and potassium stearate, sold under the name Tegin® by the company Goldschmidt (CTFA name: glyceryl stearate SE).

Preferably, a composition according to the invention comprises at least one surfactant, in particular a non-ionic surfactant, and preferably a surfactant chosen from glyceryl esters.

### Anionic Surfactant

The anionic surfactants may be chosen from alkyl ether sulfates, carboxylates, amino acid derivatives, sulfonates, isethionates, taurates, sulfosuccinates, alkylsulfoacetates, phosphates and alkyl phosphates, polypeptides, metal salts of C₁₀-C₃₀ and notably C₁₆-C₂₅ fatty acids, in particular metal stearates and behenates, alkali metal salts of cetyl phosphate, and mixtures thereof.

As alkali metal salts of cetyl phosphate, mention may notably be made of potassium cetyl phosphate. Use may notably be made of the monopotassium salt of monocetyl phosphate (INCI name: potassium cetyl phosphate) sold under the name “Amphisol K” by the company DSM Nutritional Products.

### Cationic Surfactant

The cationic surfactants may be chosen from alkylimidazolidiniums, such as isostearyl ethylimidonium ethosulfate, ammonium salts such as (C₁₂₋₃₀-alkyl)-tri(C₁₋₄-alkyl)ammonium halides such as N,N,N-trimethyl-1-docosanaminium chloride (or behentrimonium chloride).

### Amphoteric Surfactant

The compositions according to the invention may also contain one or more amphoteric surfactants, for instance N-acylamino acids such as N-alkyl aminoacetates and disodium cocoamphodiacetate, and amine oxides such as stearamine oxide, or alternatively silicone surfactants, for instance dimethicone copolyol phosphates such as the product sold under the name Pecosil PS 100® by the company Phoenix Chemical.

### Silicone Surfactant

The composition may also comprise at least one silicone surfactant. By way of example, as non-ionic surfactants with an HLB of greater than or equal to 8 at 25° C., used alone or as a mixture, mention may be made of dimethicone copolyol or dimethicone copolyol benzoate, and as non-ionic surfactants with an HLB of less than 8 at 25° C., used alone or as a mixture, mention may be made of the cyclomethicone/dimethicone copolyol mixture.

Preferably, a composition according to the invention comprises less than 2% by weight of silicone surfactant(s), in particular less than 1% by weight of silicone surfactant(s), preferably less than 0.5% by weight of silicone surfactant(s) and more preferentially is free of silicone surfactant(s).

The surfactant(s) may be present in a composition according to the invention in a proportion ranging from 0.5% to 10% by weight and preferably from 0.5% to 5% by weight, relative to the total weight of the composition.

### Gelling Agent/Thickener

Depending on the fluidity of the composition that it is desired to obtain, one or more thickeners and/or gelling agents, which are in particular hydrophilic, that is to say water-soluble or water-dispersible, may be incorporated into the composition.

Examples of hydrophilic gelling agents that may be mentioned include modified or unmodified carboxyvinyl polymers, such as the products sold under the names Carbopol (CTFA name: carbomer) and Pemulen (CTFA name: Acrylates/C10-30 alkyl acrylate crosspolymer) by the company Goodrich; polyacrylamides; optionally crosslinked and/or neutralized 2-acrylamido-2-methylpropanesulfonic acid polymers and copolymers, for instance the poly(2-acrylamido-2-methylpropanesulfonic acid) sold by the company Hoechst under the name Hostacerin AMPS (CTFA name: Ammonium polyacryldimethyltauramide); crosslinked anionic copolymers of acrylamide and of AMPS, which are in the form of a W/O emulsion, such as those sold under the name Sepigel 305 (CTFA name: Polyacrylamide/C13-14 Isoparaffin/Laureth-7) and under the name Simulgel 600 (CTFA name: Acrylamide/Sodium acryloyldimethyltaurate copolymer/Isohexadecane/Polysorbate 80) by the company SEPPIC; polysaccharide biopolymers, for instance xanthan gum, guar gum, alginates and modified celluloses, and mixtures thereof.

The amount of gelling agents depends on the desired objective.

According to one embodiment, the amount of gelling agents ranges for example from 0.01% to 10% and for example from 0.05% to 5% by weight, relative to the total weight of the composition.

### Fillers

According to one particular embodiment, a composition according to the invention may also comprise one or more fillers, in particular chosen from those conventionally used in care and/or makeup compositions.

For the purposes of the present invention, the term “fillers” is understood to mean colourless or white, mineral or organic, natural or synthetic solid particles of any form, which are in a form that is insoluble and dispersed in the medium of the composition.

Of course, these fillers are used in appropriate contents and under appropriate conditions so as not to be detrimental to the properties of the composition.

These fillers make it possible to give the composition containing them softness, a matt effect and uniformity. In addition, these fillers advantageously make it possible to combat various attacking factors such as sebum or sweat.

By way of illustration of these fillers, mention may be made of talc, mica, silica, kaolin, poly-β-alanine powder and polyethylene powder, tetrafluoroethylene polymer (Teflon®) powders, lauroyllysine, starch, boron nitride, hollow polymer microspheres such as those of polyvinylidene chloride/acrylonitrile, for instance Expancel® (Nobel Industrie), acrylic acid copolymer microspheres, silicone resin microbeads (for example Tospearls® from Toshiba), polyorganosiloxane elastomer particles, precipitated calcium carbonate, magnesium carbonate, magnesium hydrocarbonate, hydroxyapatite, barium sulfate, aluminium oxides, polyurethane powders, composite fillers, hollow silica microspheres, and glass or ceramic microcapsules. Use may also be made of particles that are in the form of hollow sphere portions, as described in patent applications JP-2003 128 788 and JP-2000 191 789.

### Colorants

A composition according to the invention may also comprise at least one particulate or non-particulate, water-soluble or water-insoluble colorant, preferably in a proportion of at least 0.0001% by weight relative to the total weight of the composition.

For obvious reasons, this amount is liable to vary significantly with regard to the intensity of the desired colour effect and of the colour intensity afforded by the colorants under consideration, and its adjustment clearly falls within the competence of a person skilled in the art.

As stated above, the colorants that are suitable for use in the invention may be water-soluble, but may also be liposoluble.

### Water-Soluble Dyes

For the purposes of the invention, the term “water-soluble colorant” means any natural or synthetic, generally organic compound, which is soluble in an aqueous phase or water-miscible solvents and which is capable of imparting colour.

As water-soluble dyes that are suitable for use in the invention, mention may be made notably of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanine (beetroot), carmine, copper chlorophyllin, methylene blue, anthocyanins (enocianin, black carrot, hibiscus, elder), caramel and riboflavin.

The water-soluble dyes are, for example, beetroot juice and caramel.

According to a preferred embodiment, the composition according to the invention comprises at least one water-soluble dye.

The water-soluble dyes may be present in a proportion of from 0.0001% to 2% by weight relative to the total weight of the composition containing them.

### Liposoluble Dyes

For the purposes of the invention, the term “liposoluble colorant” is intended to mean any natural or synthetic, generally organic compound, which is soluble in an oily phase or in solvents that are miscible with a fatty substance, and which is capable of imparting colour.

As liposoluble dyes that are suitable for use in the invention, mention may notably be made of synthetic or natural liposoluble dyes, for instance DC Red 17, DC Red 21, DC Red 27, DC Green 6, DC Yellow 11, DC Violet 2, DC Orange 5, Sudan red, carotenes (0-carotene, lycopene), xanthophylls (capsanthin, capsorubin, lutein), palm oil, Sudan brown, quinoline yellow, annatto and curcumin.

The colouring particulate materials may be present in a proportion of from 0.0001% to 10% by weight relative to the total weight of the composition containing them.

They may especially be pigments, nacres and/or particles with metallic tints.

### Pigments

The term “pigments” should be understood as meaning white or coloured, mineral or organic particles that are insoluble in an aqueous solution, which are intended to colour and/or opacify the composition containing them.

The pigments may be white or coloured, and mineral and/or organic.

As mineral pigments, mention may be made of titanium oxide, titanium dioxide, zirconium oxide, zirconium dioxide, cerium oxide or cerium dioxide and also zinc oxide, iron oxide or chromium oxide, ferric blue, manganese violet, ultramarine blue and chromium hydrate, and mixtures thereof.

They may also be pigments having a structure that may be, for example, of sericite/brown iron oxide/titanium dioxide/silica type. Such a pigment is sold, for example, under the reference Coverleaf NS or JS by the company Chemicals And Catalysts, and has a contrast ratio in the region of 30.

They may also be pigments having a structure that may be, for example, of silica microsphere type containing iron oxide. An example of a pigment having this structure is the product sold by Miyoshi under the reference PC Ball PC-LL-100 P, this pigment being constituted of silica microspheres containing yellow iron oxide.

Advantageously, the pigments may be iron oxides and/or titanium dioxides.

According to one particular embodiment, a composition according to the invention comprises less than 1% of pigments by weight.

### Nacres

The term “nacres” should be understood as meaning coloured particles of any form, which may or may not be iridescent, especially produced by certain molluscs in their shell, or alternatively synthesized, and which have a colour effect via optical interference.

The nacres can be chosen from pearlescent pigments, such as titanium oxide-coated mica covered with an iron oxide, titanium oxide-coated mica covered with bismuth oxychloride, titanium oxide-coated mica covered with chromium oxide, titanium oxide-coated mica covered with an organic dye and also pearlescent pigments based on bismuth oxychloride.

They can also be mica particles, at the surface of which are superimposed at least two successive layers of metal oxides and/or of organic colorants.

Examples of nacres that may also be mentioned include natural mica covered with titanium oxide, with iron oxide, with natural pigment or with bismuth oxychloride.

Mention may be made, among the commercially available nacres, of the Timica, Flamenco and Duochrome (on a mica base) nacres sold by Engelhard, the Timiron nacres sold by Merck, the Prestige nacres on a mica base sold by Eckart and the Sunshine synthetic mica-based nacres sold by Sun Chemical.

The nacres can more particularly have a yellow, pink, red, bronze, orangey, brown, gold and/or coppery colour or tint.

Advantageously, the nacres in accordance with the invention are micas covered with titanium dioxide or with iron oxide, and also bismuth oxychloride.

According to one particular embodiment, a composition according to the invention comprises less than 1% by weight of nacres.

Particles with a Metallic Tint

For the purposes of the present invention, the term “particles with a metallic tint” means any compound of which the nature, size, structure and surface finish allow it to reflect incident light, in particular in a non-iridescent manner.

The particles with a metallic tint which can be used in the invention are in particular chosen from particles of at least one metal and/or at least one metal derivative; particles comprising an organic or mineral substrate, made from a single material or multiple materials, at least partially covered with at least one layer with a metallic tint comprising at least one metal and/or at least one metal derivative, and mixtures of said particles.

Among the metals that may be present in said particles, mention may be made, for example, of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo and Cr, and mixtures or alloys thereof (for example bronzes and brasses) are preferred metals.

The term “metal derivatives” denotes compounds derived from metals, in particular oxides, fluorides, chlorides and sulfides.

Illustrations of these particles that may be mentioned include aluminium particles, such as those sold under the names Starbrite 1200 EAC® by the company Siberline and Metalure® by the company Eckart and glass particles coated with a metallic layer, notably those described in JP-A-09188830, JP-A-10158450, JP-A-10158541, JP-A-07258460 and JP-A-05017710.

According to one particular embodiment, a composition according to the invention comprises less than 1% of particles with a metallic tint.

### Hydrophobic Treatment of the Colorants

The pulverulent colorants as described previously may be totally or partially surface treated, with a hydrophobic agent, to make them more compatible with the oily phase of the composition of the invention, notably so that they have good wettability with oils. Thus, these treated pigments are well dispersed in the oily phase. Hydrophobically treated pigments are notably described in EP-A-1 086 683.

The hydrophobic-treatment agent may be chosen from silicones such as methicones, dimethicones and perfluoroalkylsilanes; fatty acids, such as stearic acid; metal soaps, such as aluminium dimyristate, the aluminium salt of hydrogenated tallow glutamate; perfluoroalkyl phosphates; hexafluoropropylene polyoxides; perfluoropolyethers; amino acids; N-acylamino acids or salts thereof; lecithin, isopropyl triisostearyl titanate, isostearyl sebacate, and mixtures thereof.

The term “alkyl” mentioned in the compounds cited above notably denotes an alkyl group containing from 1 to 30 carbon atoms and preferably containing from 5 to 16 carbon atoms.

### Additives

The composition in accordance with the present invention may also comprise conventional cosmetic adjuvants notably chosen from active agents, softeners, humectants, opacifiers, emollients, silicones, antifoams, fragrances, polar additives, preservatives, in particular phenoxyethanol, polymers, for example film-forming polymers, propellants, dispersants, anti-pollution agents, chelating agents, basifying or acidifying agents or any other ingredient commonly used in the cosmetic and/or dermatological field.

Among the acidifying agents, examples that may be mentioned include mineral or organic acids, for instance hydrochloric acid, orthophosphoric acid, sulfuric acid, carboxylic acids, for instance acetic acid, tartaric acid, citric acid or lactic acid, and sulfonic acids.

Among the basifying agents, examples that may be mentioned include aqueous ammonia, alkali metal carbonates, alkanolamines, such as mono-, di- and triethanolamines and derivatives thereof, sodium hydroxide or potassium hydroxide.

Preferably, the cosmetic composition comprises one or more basifying agents chosen from alkanolamines, in particular triethanolamine, and sodium hydroxide.

In the case of a direct emulsion, the pH of the composition in accordance with the invention is generally between 3 and 12 approximately, preferably between 5 and 11 approximately and even more particularly from 5.5 to 8.

Among the active agents for caring for keratin materials such as the skin, the lips, the scalp, the hair, the eyelashes or the nails, mention may be made for example of vitamins and derivatives or precursors thereof, alone or as mixtures; antioxidants; free-radical scavengers; anti-pollutants; self-tanning agents; anti-glycation agents; calmatives; deodorant agents; essential oils; NO-synthase inhibitors; agents for stimulating the synthesis of dermal or epidermal macromolecules and/or for preventing degradation thereof; agents for stimulating fibroblast proliferation; agents for stimulating keratinocyte proliferation; muscle relaxants; refreshing agents; tensioning agents; matifying agents; depigmenting agents; propigmenting agents; keratolytic agents; desquamating agents; moisturising agents; anti-inflammatories; antimicrobials; thinning agents; agents which act on cell energy metabolism; insect repellents; substance P antagonists or CRGP antagonists; agents for preventing hair loss; antiwrinkle agents; anti-ageing agents.

A person skilled in the art will choose said active agent(s) as a function of the effect desired on the skin, the hair, the eyelashes, the eyebrows or the nails.

Needless to say, a person skilled in the art will take care to select this or these optional additional compound(s), and/or the amount thereof, such that the advantageous properties of a composition according to the invention are not, or are not substantially, adversely affected by the envisioned addition.

### Composition

A composition according to the invention may be in the form of an emulsion, for example an oil-in-water (O/W) or water-in-oil (W/O) emulsion, of a gel, for example an oil-in-water or water-in-oil emulsified gel, of an aqueous composition, or else in the form of a composition of gel/gel type.

According to one embodiment, the composition according to the present invention is an emulsion.

According to one variant, the composition according to the invention is a simple emulsion.

According to one variant, the composition according to the invention is a multiple emulsion.

It may be a triple emulsion.

According to one variant, the composition according to the invention is a direct simple emulsion. Thus, according to one embodiment, the composition according to the present invention is an oil-in-water (O/W) emulsion.

According to one variant, the composition according to the invention is an inverse simple emulsion. Thus, according to one embodiment, the composition according to the present invention is a water-in-oil (W/O) emulsion.

According to one variant, the composition according to the invention is an aqueous formulation of aqueous gel or solution type.

Preferably, the composition is an emulsion, especially a water-in-oil or oil-in-water emulsion.

In the case of compositions in the form of oil-in-water or water-in-oil emulsions, the emulsification processes which can be used are of the paddle or propeller, rotor-stator and HPH type.

In order to obtain stable emulsions with a low content of polymer (oil/polymer ratio>25), it is possible to prepare the dispersion in concentrated phase and then to dilute the dispersion with the remainder of the aqueous phase.

It is also possible, via HPH (between 50 and 800 bar), to obtain stable dispersions with drop sizes that can be as small as 100 nm.

The composition according to the invention may be more or less fluid and may have the appearance of a white or coloured cream, an ointment, a milk, a lotion, a serum, a paste or a foam. It may optionally be applied to the skin in aerosol form. It may also be in solid form, for example in the form of a stick.

A composition according to the invention may be in the form of a cosmetic composition for caring for and/or making up keratin materials, preferably a cosmetic composition for caring for keratin materials, in particular of the body or of the face, preferably of the face.

The compositions according to the invention find their application in a large number of treatments, in particular cosmetic treatments, for the skin, the lips and the hair, including the scalp, in particular for protecting and/or caring for the skin, the lips and/or the hair, and/or for making up the skin and/or the lips.

Another subject of the present invention is constituted of the use of the compositions according to the invention as defined above in the manufacture of products for the cosmetic treatment of the skin, lips, nails, hair, eyelashes, eyebrows and/or scalp, in particular of care products, sun protection products and makeup products.

The compositions according to the invention may be in the form of products for caring for the skin or semi-mucous membranes, such as a protective or cosmetic care composition for the face, for the lips, for the hands, for the feet, for the anatomical folds or for the body (for example, day creams, night cream, day serum, night serum, makeup-removing cream, makeup base, antisun composition, protective or care body milk, aftersun milk, skincare or scalp-care lotion, gel or foam, serum, mask, or aftershave composition).

The cosmetic compositions according to the invention may be used, for example, as care products and/or sun protection products for the face and/or the body, of liquid to semiliquid consistency, such as lotions, milks, more or less rich creams, gels and cream-gels. They can optionally be packaged as an aerosol and be provided in the form of a foam or of a spray.

The compositions according to the invention in the form of vaporizable fluid lotions in accordance with the invention are applied to the skin or hair in the form of fine particles by means of pressurizing devices. The devices in accordance with the invention are well known to those skilled in the art and include non-aerosol pumps or “atomizers”, aerosol containers comprising a propellant and aerosol pumps using compressed air as propellant. These devices are described in patents U.S. Pat. Nos. 4,077,441 and 4,850,517.

The compositions packaged in aerosol form in accordance with the invention generally contain conventional propellants, for instance hydrofluoro compounds, dichlorodifluoromethane, difluoroethane, dimethyl ether, isobutane, n-butane, propane or trichlorofluoromethane. They are preferably present in amounts ranging from 15% to 50% by weight relative to the total weight of the composition.

The composition may be applied to the skin by hand or using an applicator.

According to another aspect, the invention also relates to a cosmetic assembly comprising:
i) a container delimiting one or more compartment(s), said container being closed by a closing member and optionally being unsealed; and
ii) a makeup and/or care composition in accordance with the invention placed inside said compartment(s).

The container can, for example, be in the form of a pot or a case.

The closing member may be in the form of a lid comprising a cap mounted so as to be able to move by translation or by pivoting relative to the container housing said makeup and/or care composition(s).

Such compositions are notably prepared according to the general knowledge of a person skilled in the art.

Such compositions as defined previously may especially be employed for a cosmetic use according to the invention.

Thus, the invention also relates to the use of a composition according to the invention for caring for and/or making up keratin materials, preferably for caring for keratin materials, in particular the skin of the body and/or of the face.

The invention also relates to a cosmetic process for making up and/or caring for keratin materials, in particular the skin, comprising at least one step of applying a composition as defined previously to said keratin materials.

Preferably, the invention also relates to a cosmetic process for caring for keratin materials, in particular of the body and/or of the face, comprising at least one step of applying a composition as defined above to said keratin materials.

The cosmetic processes for making up and/or caring for keratin materials, in particular the skin, are non-therapeutic.

In particular, the present invention relates to the non-therapeutic cosmetic use of a composition as described above, for protecting keratin materials, in particular the skin and/or the lips and/or the hair against solar radiation.

The invention also relates to the non-therapeutic cosmetic use of a composition as defined above, for combating or preventing the signs of photo-induced premature ageing of keratin materials, in particular the skin and/or the lips and/or the hair.

According to one embodiment, the invention also relates to the use of a composition according to the invention for reducing the pigmentation induced by UVA rays, UVB rays and long UVA rays.

The present invention also relates to the cosmetic use of a composition as defined above, for preventing darkening of the skin and/or improving the colour and/or the uniformity of the complexion.

In addition, the present invention relates to the cosmetic use of a composition as defined above, for preventing premature ageing of keratin materials, in particular the skin, especially on the face, the neckline, the arms, the hands and/or the shoulders, in particular the signs of skin ageing of actinic origin, such as photoageing.

The present invention also relates to the cosmetic use of a composition as defined above to prevent a loss of firmness and/or elasticity and/or tonicity and/or suppleness of the skin, the formation of wrinkles and fine lines, a dull complexion, and/or a wizened appearance of the skin.

The present invention also relates to a non-therapeutic cosmetic process for protecting keratin materials, in particular the skin and/or the lips and/or the hair against solar radiation, comprising the application, to the surface of the keratin material, of at least one composition according to the invention.

It also relates to a non-therapeutic cosmetic process for preventing and/or treating the signs of ageing of a keratin material, comprising the application, to the surface of the keratin material, of at least one composition according to the invention.

For the purposes of the present invention, the term “preventing” is understood to mean, at least partly, reducing the risk of occurrence of a given phenomenon.

Throughout the description, including the claims, the expression “including a” should be understood as being synonymous with “including at least one”, unless otherwise specified.

The expressions “between . . . and . . . ”, “comprises from . . . to . . . ”, “formed from . . . to . . . ” and “ranging from . . . to . . . ” should be understood as being inclusive of the limits, unless otherwise specified.

In the description and the examples, unless otherwise indicated, the percentages are percentages by weight. The percentages are thus expressed by weight, with respect to the total weight of the composition. The temperature is expressed in degrees Celsius, unless otherwise indicated, and the pressure is atmospheric pressure, unless otherwise indicated.

The invention is illustrated in greater detail by the non-limiting examples presented below.

### EXAMPLE

### Example A: Preparation of Merocyanines According to the Invention

### Example A1: Preparation of Compound (A)

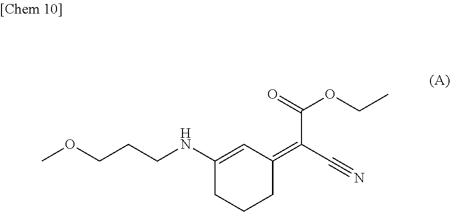 [SMILES: CCOC(=O)/C(C#N)=C1\C=C(NCCCOC)CCC1]

122.23 g of 3-[(3-methoxypropyl)amino]-2-cyclohexen-1-one were alkylated with dimethyl sulfate or alternatively with diethyl sulfate and treated with 75.45 g of ethyl cyanoacetate in approximately equimolar proportions in the presence of a base and optionally of a solvent.

The following base/solvent combinations were used:

TABLE 2

| Example | Base | Solvent |
|---|---|---|
| Example A1.1 | DBU (1,8-diazabicyclo[5.4.0] | dimethylacetamide |
| | undec-7-ene) | |
| Example A1.2 | triethylamine | isopropanol |
| Example A1.3 | 3-methoxypropylamine | isopropanol |
| Example A1.4 | 3-methoxypropylamine | tert-amyl alcohol |
| Example A1.5 | 3-methoxypropylamine | toluene |
| Example A1.6 | 3-methoxypropylamine | dimethylformamide |
| Example A1.7 | 3-methoxypropylamine | Without solvent |
| Example A1.8 | N-morpholine | isopropanol |

The completion of the alkylation reaction could be monitored, for example, via methods such as TLC, GC or HPLC.162.30 g of compound (14) were obtained in the form of a brown oil. After crystallization, the product was obtained in the form of yellowish crystals.

Melting point: 92.7° C.

### Example A2: Preparation of Compound (B)

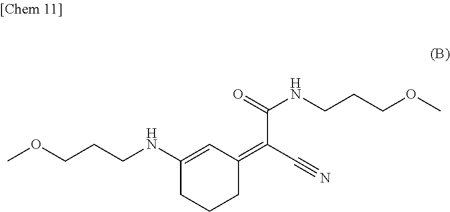

101.00 g of 3-[(3-methoxypropyl)amino]-2-cyclohexen-1-one were alkylated with dimethyl sulfate or alternatively with diethyl sulfate and treated with 86.00 g of 2-cyano-N-(3-methoxypropyl)acetamide in approximately equimolar proportions in the presence of a base and optionally of a solvent.

The following base/solvent combinations were used:

TABLE 3

| Example | Base | Solvent |
|---|---|---|
| Example A2.1 | DBU (1,8-diazabicyclo[5.4.0] | dimethylacetamide |
| | undec-7-ene) | |
| Example A2.2 | triethylamine | isopropanol |
| Example A2.3 | 3-methoxypropylamine | isopropanol |
| Example A2.4 | 3-methoxypropylamine | tert-amyl alcohol |
| Example A2.5 | 3-methoxypropylamine | toluene |
| Example A2.6 | 3-methoxypropylamine | dimethylformamide |
| Example A2.7 | 3-methoxypropylamine | without solvent |

The crude product (B) was obtained in the form of a dark brown oil. After silica gel column chromatography (eluent: 99/1 toluene/methanol), 81.8 g of product were obtained in the form of yellowish crystals.

Melting point: 84.7-85.3° C.

### Example A3: Preparation of Compound (D)

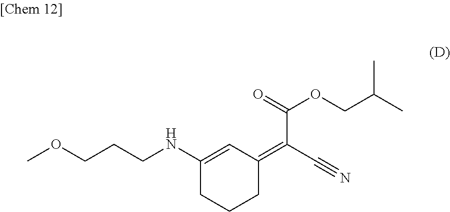 [SMILES: COCCCNC1=C/C(=C(/C#N)C(=O)OCC(C)C)CCC1]

13.09 g of 3-[(3-methoxypropyl)amino]-2-cyclohexen-1-one were alkylated with dimethyl sulfate or alternatively with diethyl sulfate and treated with 10.12 g of isobutyl cyanoacetate in the presence of a base and optionally of a solvent.

The following base/solvent combinations were used:

TABLE 4

| Example | Base | Solvent |
|---|---|---|
| Example A3.1 | DBU (1,8-diazabicyclo[5.4.0] | dimethylacetamide |
| | undec-7-ene) | |
| Example A3.2 | triethylamine | isopropanol |
| Example A3.3 | 3-methoxypropylamine | isopropanol |
| Example A3.4 | N-methylmorpholine | tert-amyl alcohol |
| Example A3.5 | 3-methoxypropylamine | toluene |
| Example A3.6 | 3-methoxypropylamine | dimethylformamide |
| Example A3.7 | 3-methoxypropylamine | without solvent |

15.97 g of crude product (27) were obtained in the form of a dark brown oil. After silica gel column chromatography (eluent: toluene/acetone), 13.46 g of product were obtained in the form of yellowish crystals.

Melting point: 96.3° C.

### Example A4: Preparation of Compound (C)

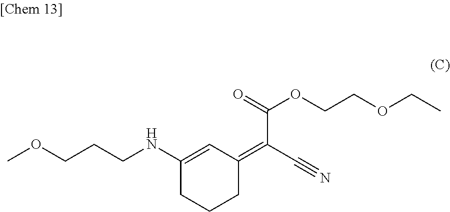 [SMILES: CCOCCOC(=O)/C(C#N)=C1\C=C(NCCCOC)CCC1]

148.4 g of 3-[(3-methoxypropyl)amino]-2-cyclohexen-1-one were alkylated with dimethyl sulfate or alternatively with diethyl sulfate and treated with 130.00 g of 2-ethoxyethyl cyanoacetate in the presence of an organic base and of a solvent.

The following base/solvent combinations were used:

TABLE 5

| Example | Base | Solvent |
|---|---|---|
| Example A4.1 | DBU (1,8-diazabicyclo[5.4.0] | dimethylacetamide |
| | undec-7-ene) | |
| Example A4.2 | triethylamine | isopropanol |
| Example A4.3 | 3-methoxypropylamine | isopropanol |
| Example A4.4 | N-methylmorpholine | tert-amyl alcohol |
| Example A4.5 | 3-methoxypropylamine | toluene |
| Example A4.6 | 3-methoxypropylamine | dimethylformamide |
| Example A4.7 | 3-methoxypropylamine | without solvent |

### Example 1: Tests of Solubilization of Methoxypropylamino Cyclohexenylidene Ethoxyethylcyanoacetate (C) Prepared in Example A4 in Saturated Simplex Solutions

Simplex solutions comprising the Methoxypropylamino Cyclohexenylidene Ethoxyethylcyanoacetate screening agent in excess in various solvents and mixtures of solvents are prepared, using the weight proportions as detailed in the table below. The values are expressed as weight percentages relative to the total weight of the composition.

TABLE 6

| | Formula 1 | | | | |
|---|---|---|---|---|---|
| | Outside the | Formula 2 | Formula 3 | Formula 4 | Formula 5 |
| Compounds | invention | Invention | Invention | Invention | Invention |
| Water | 95.24 | 90.48 | 93.33 | 85.72 | 77.14 |
| Methoxypropylamino | 4.76 | 4.76 | 4.76 | 4.76 | 4.76 |
| Cyclohexenylidene | | | | | |
| Ethoxyethylcyanoacetate | | | | | |
| (according to Example A) | | | | | |
| Nicotinamide | | 4.76 | | 4.76 | 4.76 |
| (DSM Nutritional Products) | | | | | |
| Caffeine (BASF) | | | 1.91 | 4.76 | 4.76 |
| Dipropylene Glycol (DOW) | | | | | 8.58 |

The Methoxypropylamino Cyclohexenylidene Ethoxyethylcyanoacetate screening agent is assayed in order to calculate its solubility in the various solvents and mixtures of solvents.

In each of the formulae, the screening agent is present at 5% in excess in order to carry out solubility measurements at comparative saturation.

The solutions are filtered over 0.2 m before the assay. The assay is carried out 72 hours after the preparation of the compositions, at room temperature.

For the formula 4, an additional assay was carried out 2 months after the preparation of the composition, at room temperature.

The assay was carried out by HPLC/UV under the following conditions: System: Acquity UPLC, BEH Shield RP18 column, 1.7 μm, (2.1 mm×100 mm) from Waters, eluent: methanol/H₃PO₄ acidified water, elution gradient 0.1%. The sample is prepared by dilution in methanol.

The results are detailed in the table below.

TABLE 7

| | Formula 1 | | | | |
|---|---|---|---|---|---|
| | Outside | | | | |
| | the | Formula 2 | Formula 3 | Formula 4 | Formula 5 |
| | invention | Invention | Invention | Invention | Invention |
| 72 hours at | 0.056% | 0.34% | 0.26% | 0.99% | 1.38% |
| room | | | | | |
| temperature | | | | | |
| 2 months | / | / | / | 0.91% | / |
| at room | | | | | |
| temperature | | | | | |

The solubility measurements show that the aqueous solubility of the Methoxypropylamino Cyclohexenylidene Ethoxyethylcyanoacetate screening agent (C) is increased in the compositions according to the invention which comprise nicotinamide and/or caffeine, compared to formula 1 which does not comprise a hydrotrope.

This solubility is verified over time since it remains stable two months after preparation of the composition, as is demonstrated by the measurement carried out on composition 4 according to the invention.

The solubility in an aqueous medium of the compound (C) was also verified in the combined presence of a single hydrotrope and of one or more organic solvents, of which the nature and the weight proportions are detailed in Table 5 below. The values are expressed as weight percentages relative to the total weight of the composition.

Formulations 1A to G were prepared according to the following procedure:
Solubilizing compound 25 in propylene carbonate by heating at 65° C. until a clear mixture is obtained.
Leaving to cool to around 45° C. and then introducing the other ingredients finishing with the addition of ethanol.
Heating the mixture again at 65° C.
In the case where hydrotropes have been added (Examples 2, 3, 5, 6 and 7), creating a vortex until a clear mixture is obtained.
Leaving at 65° C. for 1 h then returning to room temperature for 24 h.
Centrifuging the mixture at 4000 rpm for 10 min to accelerate the phase separation.
The mixture is then stored at room temperature and the phase separation is observed after 2 weeks.

TABLE 8

| | Formula A | | |
|---|---|---|---|
| | Outside the | Formula B | Formula C |
| Ingredients | invention | Invention | Invention |
| Compound (C) | 3 | 3 | 3 |
| Propylene carbonate | 19.4 | 14.55 | 14.55 |
| Nicotinamide | 0 | 4.85 | 0 |
| Sodium salicylate | 0 | 0 | 4.85 |
| Water | 38.8 | 38.8 | 38.8 |
| Miglyol 810 | 38.8 | 38.8 | 38.8 |
| Number of aqueous phases | 2 | 1 | 1 |

TABLE 9

| | Formula D | | | |
|---|---|---|---|---|
| | Outside the | Formula E | Formula F | Formula G |
| Ingredients | invention | Invention | Invention | Invention |
| Compound (C) | 3 | 3 | 3 | 3 |
| Propylene carbonate | 12.125 | 12.125 | 12.125 | 12.125 |
| (Merck) | | | | |
| Ethanol | 7.275 | 4.85 | 4.85 | 4.85 |
| (Sigma Aldrich) | | | | |
| Nicotinamide | | 2.425 | | |
| (ThermoFisher GmbH) | | | | |
| Sodium salicylate | | | 2.425 | |
| (Sigma Aldrich) | | | | |
| Caffeine | | | | 2.425 |
| (Aliacura) | | | | |
| Water | 38.8 | 38.8 | 38.8 | 38.8 |
| Miglyol 810 | 38.8 | 38.8 | 38.8 | 38.8 |
| (Sasol) | | | | |
| Number of aqueous | 2 | 1 | 1 | 1 |
| phases | | | | |

It is observed that the lack of solubility of compound (C) either in water (Formula A) or in an aqueous mixture containing at least one organic solvent (Formula D) is overcome by the presence of a hydrotrope such as for example nicotinamide or sodium salicylate or caffeine.

### Example 2: Composition 6 According to the Invention

Composition 6 according to the invention in gel form is prepared using the weight proportions as detailed in the table below. The values are expressed as weight percentages relative to the total weight of the composition.

TABLE 10

| | | Formula 6 |
|---|---|---|
| Phase | Compounds (INCI) | Invention |
| A | Water | 76.4 |
| | Dipropylene Glycol (Dow) | 10 |
| | Niacinamide (DSM Nutritional Products) | 5 |
| | Caffeine (BASF) | 5 |
| | Methoxypropylamino Cyclohexenylidene | 1.3 |
| | Ethoxyethylcyanoacetate | |
| | (according to Example A) | |
| B | Phenoxyethanol (Toho Chemicals) | 0.7 |
| | Caprylyl Glycol (Kolon Life Science) | 0.2 |
| C1 | Carbomer | 0.3 |
| | (Acrypol 980 from Corel Pharma Chem) | |
| | Xanthan Gum (CP Kelco) | 0.1 |
| C2 | Sodium Hydroxide | 0 |
| | (PT. Mulia Agung Chemindo) | |
| D | Water | 1 |

The composition is prepared according to the following procedure:

In a beaker with a Rayneri paddle, the water of phase A, the dipropylene glycol and the niacinamide are mixed. When the solution is clear, the caffeine is introduced, then the Methoxypropylamino Cyclohexenylidene Ethoxyethylcyanoacetate is introduced, leaving the solution stirred and heated at 40° C. Once the solution is clear, the preservatives are added, then the gelling agents are introduced, and the composition is left to swell. The pH is then measured, and the composition is neutralized with NaOH to obtain a pH equal to 6. The water of phase D is then added.

The composition obtained is advantageously a single-phase composition.

### Example 3: Compositions 7 and 8 According to the Invention

Compositions 7 and 8 according to the invention in the form of oil-in-water emulsions are prepared using the weight proportions as detailed in the table below. The values are expressed as weight percentages relative to the total weight of the composition.

TABLE 11

| | | Formula 7 |
|---|---|---|
| Phase | Compounds (INCI) | Invention |
| A | Sucrose Tristearate (Ryoto Sugar Ester S270 from | 1.08 |
| | Mitsubishi-Kagaku Foods) | |
| | PEG-40 Stearate (Tego Acid S40 P MB from Evonik | 1.57 |
| | Goldschmidt) | |
| | Glyceryl Stearate (DUB GMS/MB from Stearinerie | 2 |
| | Dubois) | |
| | C₁₅₋₁₉ Alkane (Emogreen L19 from Seppic) | 7.9 |
| B1 | Water | 59.65 |
| | Dipropylene Glycol (Dow) | 8 |
| | Niacinamide (DSM Nutritional Products) | 4 |
| | Caffeine (BASF) | 4 |
| | Methoxypropylamino Cyclohexenylidene | 1 |
| | Ethoxyethylcyanoacetate (according to Example A) | |
| | Trisodium Ethylenediamine Disuccinate | 0.25 |
| | (Natrlquest E30 from Innospec Active Chemicals) | |
| | Hydroxyacetophenone (Symsave H O from Symrise) | 0.5 |
| | Pentylene Glycol (A-Leen 5 from Mina solve) | 3 |
| B2 | Sodium Hyaluronate (Cristalhyal LO from Soliance | 0.5 |
| | (Givaudan)) | |
| | Sodium Polyacrylate (Cosmedia SP from BASF) | 0.5 |
| B3 | Gemini Surfactant (Asahi Kasei) | 0.35 |
| C | Isododecane (Ineos) | 1.7 |
| | Ammonium Polyacryloyldimethyl Taurate | 1.5 |
| | (Hostacerin AMPS from Clariant) | |
| E | Lauroyl Lysine (Ajinomoto) | 2 |
| | Boron Nitride | 0.5 |

Composition 7 is prepared according to the following procedure:

The components of phase B1 are mixed and homogenized for 10 minutes at 70° C. in an emulsifier with a Rayneri paddle. The components of phase A at 60° C. are added for emulsification, the composition is left stirring for 10 minutes. The components of phases B2, B3 and C are added at 40° C. for gelation. The composition is left stirring for 15 minutes. The composition is cooled to 30° C. The fillers of phase E are incorporated and the composition is left stirring for 15 minutes.

TABLE 12

| | | Formula 8 |
|---|---|---|
| Phase | Compounds (INCI) | Invention |
| A | Niacinamide (DSM Nutritional Products) | 4 |
| | Dipropylene Glycol (Dow) | 8 |
| | Phenoxyethanol (Toho Chemicals) | 0.4 |
| | Water | 66.6 |
| | Caffeine (BASF) | 4 |
| | Trisodium Ethylenediamine Disuccinate | 0.2 |
| | (Natrlquest E30 from Innospec Active Chemicals) | |
| | Methoxypropylamino Cyclohexenylidene | 1 |
| | Ethoxyethylcyanoacetate (according to Example A) | |
| B1 | Glyceryl Stearate (and) PEG-100 Stearate | 2.5 |
| | (Tego Care 180 MB from Evonik Goldschmidt) | |
| | Cetyl Alcohol (Lanette 16 from BASF) | 2 |
| | Caprylyl Glycol (Kolon Life Science) | 0.3 |
| | Squalane (Neossance Squalane from Amyris) | 6 |
| B2 | Tocopheryl Acetate (DL Alpha Tocopheryl | 0.5 |
| | Acetate from DSM Nutritional Products) | |
| C | Xanthan Gum (CP Kelco) | 0.25 |
| | Sodium Hyaluronate (Cristalhyal LO | 0.05 |
| | from Soliance (Givaudan)) | |
| | Ammonium Polyacryloyldimethyl Taurate | 0.8 |
| | (Hostacerin AMPS from Clariant) | |
| D | Biosaccharide Gum GUM-1 | 3.4 |
| | (Fucogel 1.5P from Solabia) | |

Composition 8 is prepared according to the following procedure: In a beaker with a Rayneri paddle, the water, the dipropylene glycol, the phenoxyethanol and the niacinamide are mixed. When the solution is clear, the caffeine is introduced, then the Methoxypropylamino Cyclohexenylidene Ethoxyethylcyanoacetate is introduced, leaving the solution stirred and heated at 40° C. Once the solution is clear, the compounds of phase B1, melted beforehand at 55° C., are added for emulsification, and then the components of phase B2. The components of phase C are introduce, then the components of phase D, and the composition is left to swell.

Compositions 7 and 8 are both single-phase compositions. In other words, the compound (C) is formulated therein in solute form.

### Example 4: Composition 9

Composition 9 is prepared using weight proportions as detailed in the table below. The values are expressed as weight percentages relative to the total weight of the composition.

TABLE 13

| | | Formula |
|---|---|---|
| | | 9 |
| Phase | Compounds (INCI) | Invention |
| A1 | Water | 20 |
| | Terephthalylidene dicamphor sulfonic acid | 0.9 |
| | (Mexoryl SX from Noveal) | |
| | Triethanolamine | 0.35 |
| | Nicotinamide (ThermoFisher) | 2.5 |
| | Sodium salicylate (Sigma Aldrich) | 0.5 |
| A2 | Xanthan gum | 0.1 |
| | Glycerol | 6 |
| | Dipropylene glycol (CABB Group GmbH) | 5 |
| | Acrylates/c10-30 alkyl acrylate crosspolymer | 0.25 |
| | (Pemulen TR-1 Polymer from Lubrizol) | |
| | Water | 22 |
| B1 | Stearic acid | 1.5 |
| | Glyceryl stearate (and) peg-100 stearate | 1.3 |
| | (Arlacel 165-FP-PA-(SG) from Croda) | |
| | Homosalate | 6 |
| | Butyl methoxydibenzoylmethane | 4 |
| | (Parsol 1789 from DSM Nutritional Products) | |
| | Ethylhexyl triazone | 1.8 |
| | (Uvinul T150 from BASF) | |
| | Ethylhexyl salicylate | 5 |
| | (Parsol EHS from DSM Nutritional Products) | |
| | Isopropyl palmitate (BASF) | 7 |
| | Diisopropyl sebacate | 1 |
| | (Dub Dis from Stearinerie Dubois) | |
| | Caprylyl glycol | 0.4 |
| B2 | Potassium cetyl phosphate | 1 |
| | (Amphisol K from DSM | |
| | Nutritional Products) | |
| | pH adjuster | qs pH 6.3 |
| C1 | Propylene carbonate (Merck) | 6 |
| | Compound C | 3 |
| C2 | Ethanol | 4 |

### Procedure:

Phases A1, B1 and C are prepared separately by mixing the ingredients which make up each phase, heating at 65° C. under magnetic stirring, until a clear mixture is obtained.
The ingredients of phase B2 are introduced under stirring into phase B1 until a fine dispersion of potassium cetyl phosphate is obtained.
Phase A2 is prepared by dispersing the gelling agents in glycols, before adding water and mixing with an Ultraturrax mixer at 27 000 rpm for 5 min.
Phase C is prepared by introducing compound 25 into the propylene carbonate under mechanical stirring and while heating at 65° C. until a clear mixture is obtained. The ethanol is then incorporated once the mixture has returned to room temperature.
Phases A1 and A2 are introduced into the phase B1+B2 by mixing with the Ultraturrax mixer at 27 000 rpm for 5 min to obtain an emulsion. Phase C is then incorporated by mixing with the Ultraturrax mixer at 27 000 rpm for 5 min.

Composition 9 is advantageously an emulsion.

1. Cosmetic or dermatological composition, in particular for making up and/or for caring for keratin materials, comprising at least an aqueous phase and:
at least one merocyanine of formula (I) below and also the E/E- or E/Z-geometric isomer forms thereof: 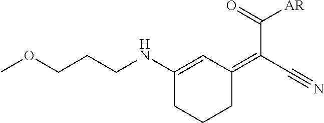
in which:
A is —O— or —NH;
R is a C₁-C₂₂ alkyl group, a C₂-C₂₂ alkenyl group, a C₂-C₂₂ alkynyl group, a C₃-C₂₂ cycloalkyl group or a C₃-C₂₂ cycloalkenyl group, it being possible for said groups to be interrupted by one or more 0; and
at least one hydrotrope chosen from nicotinamide, caffeine, salicylic acid salts, the sodium salt of pyroglutamic acid (sodium PCA), sodium 1,3-benzenedisulfonate, sodium benzoate, sodium 4-pyridinecarboxylate, sodium benzenesulfonate, sodium p-toluenesulfonate (NaPTS), sodium butyl monoglycol sulfate (NaBMGS), 4-aminobenzoic acid HCl, sodium cumene sulfonate, N,N-diethyl nicotinamide, N-picolyl nicotinamide, N-allyl nicotinamide, 2-methacryloyloxyethyl phosphorylcholine, resorcinol, pyrogallol, N-picolylacetamide, procaine HCl, proline HCl, pyridine, 3-picolylamine, sodium ibuprofen, sodium xylene sulfonate (SXS), ethyl carbamate, pyridoxal hydrochloride, sodium benzoate, N,N-dimethylacetamide, N-methylacetamide, isoniazid, and mixtures thereof.

2. Composition according to the preceding claim, comprising at least as merocyanine of formula (I), at least one compound chosen from the following compounds and also the E/E- or E/Z-geometric isomer forms thereof:

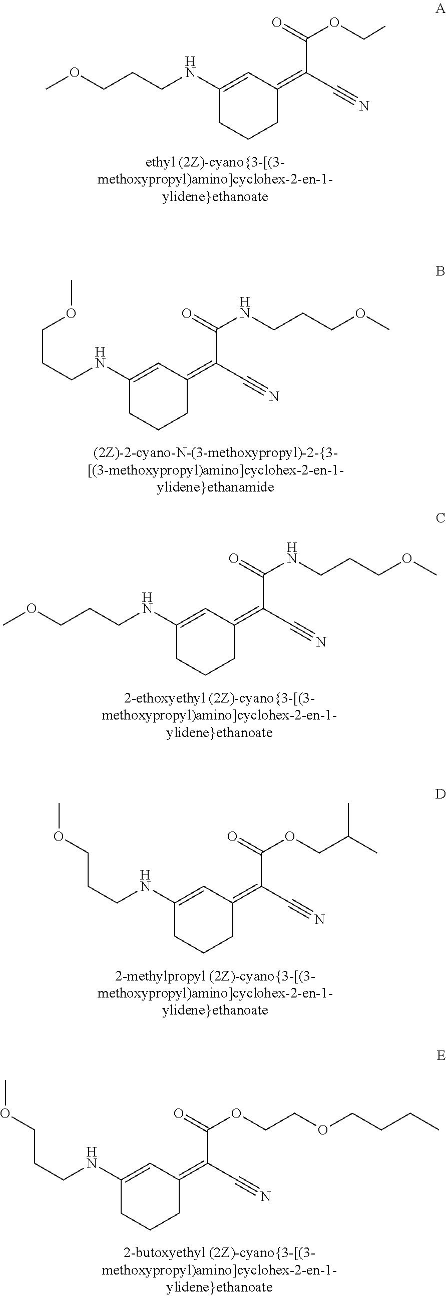

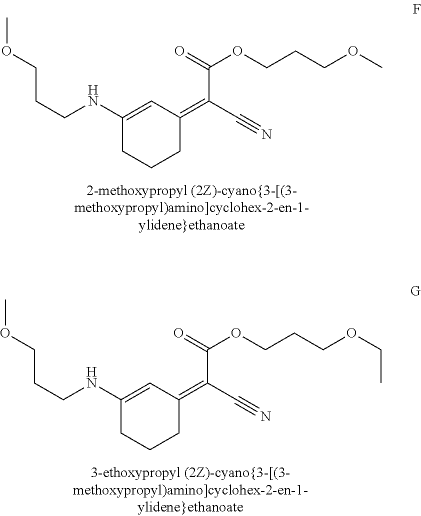 [SMILES: COCCCNC1=C/C(=C(/C#N)C(=O)OCCCOC)CCC1 ; CCOCCCOC(=O)/C(C#N)=C1\C=C(NCCCOC)CCC1]

3. Composition according to either one of the preceding claims, in which the merocyanine of formula (I) is the compound 2-ethoxyethyl (2Z)-cyano{3-[(3-methoxypropyl)amino]cyclohex-2-en-1-ylidene}ethanoate (C) in its E/Z geometric configuration having the following structure: 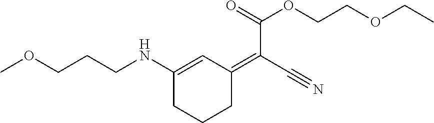 [SMILES: CCOCCOC(=O)/C(C#N)=C1\C=C(NCCCOC)CCC1]
and/or in its E/E geometric configuration having the following structure:

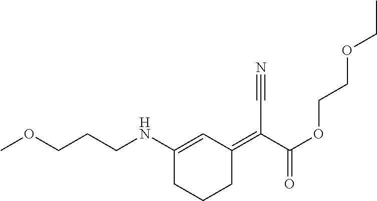 [SMILES: CCOCCOC(=O)/C(C#N)=C1/C=C(NCCCOC)CCC1]

4. Composition according to any one of the preceding claims, in which the content of merocyanine(s) of formula (I) ranges from 0.1% to 25% by weight, preferentially from 0.2% to 20% by weight, more preferentially from 0.5% to 10% by weight, and even better still from 1% to 5% by weight, relative to the total weight of the composition.

5. Composition according to any one of the preceding claims, comprising at least one hydrotrope chosen from nicotinamide, caffeine, and mixtures thereof.

6. Composition according to the preceding claim, comprising at least one additional hydrotrope chosen from salicylic acid salts, the sodium salt of pyroglutamic acid (sodium PCA), sodium 1,3-benzenedisulfonate, sodium benzoate, sodium 4-pyridinecarboxylate, sodium benzenesulfonate, sodium p-toluenesulfonate (NaPTS), sodium butyl monoglycol sulfate (NaBMGS), 4-aminobenzoic acid HCl, sodium cumene sulfonate, N,N-diethyl nicotinamide, N-picolyl nicotinamide, N-allyl nicotinamide, 2-methacryloyloxyethyl phosphorylcholine, resorcinol, pyrogallol, N-picolylacetamide, procaine HCl, proline HCl, pyridine, 3-picolylamine, sodium ibuprofen, sodium xylene sulfonate (SXS), ethyl carbamate, pyridoxal hydrochloride, sodium benzoate, N,N-dimethylacetamide, N-methylacetamide, isoniazid, and mixtures thereof, and preferably at least one salicylic acid salt.

7. Composition according to any one of the preceding claims, comprising at least nicotinamide as hydrotrope.

8. Composition according to any one of the preceding claims, in which the content of hydrotropes(s) ranges from 0.1% to 20% by weight, in particular from 0.1% to 10% by weight, preferably from 0.5% to 10% by weight, notably from 0.5% to 3% by weight, relative to the total weight of the composition.

9. Composition according to any one of the preceding claims, in which the merocyanine(s) of formula (I)/hydrotrope(s) weight ratio ranges from 0.01 to 8, preferably from 0.02 to 5, in particular from 0.05 to 3, and more particularly from 0.1 to 1.5.

10. Composition according to any one of the preceding claims, comprising at least two different hydrotropes chosen from nicotinamide, caffeine, and sodium salicylate, and the merocyanine(s) of formula (I)/hydrotrope(s) weight ratio ranging from 0.01 to 5, preferably from 0.02 to 3, in particular from 0.05 to 2, and more preferentially from 0.1 to 1.5.

11. Composition according to any one of the preceding claims, comprising two different hydrotropes chosen from nicotinamide and caffeine, and the weight ratio between these two hydrotropes ranging from 0.2 to 5, in particular from 0.5 to 3, and more preferentially from 0.8 to 1.2.

12. Composition according to any one of the preceding claims, comprising at least one alcohol, in particular a monoalcohol, and preferably ethanol.

13. Composition according to any one of the preceding claims, further comprising at least one alkylene carbonate and preferably propylene carbonate.

14. Composition according to any one of the preceding claims, comprising at least one polyol, in particular chosen from ethylene glycol, pentaerythritol, trimethylolpropane, pentylene glycol, propylene glycol, dipropylene glycol (DPG), caprylyl glycol, glycerol, polyglycerols, polyethylene glycols (PEG), and mixtures thereof, in particular chosen from propylene glycol, pentylene glycol, dipropylene glycol, and mixtures thereof, and preferably is dipropylene glycol.

15. Composition according to any one of the preceding claims, comprising at least one fatty phase, preferably ranging from 0.5% to 80% by weight, preferably from 1% to 40% by weight and more preferentially from 3% to 20% by weight relative to the total weight of the composition.

16. Composition according to any one of the preceding claims, further comprising at least one additional UV-screening agent different from the merocyanines of formula (I).

17. Composition according to any one of the preceding claims, characterized in that it is a cosmetic composition for caring for keratin materials, in particular of the body or of the face, preferably of the face.

18. Cosmetic process for caring for keratin materials, in particular of the body and/or of the face, comprising at least one step of applying a composition as defined in any one of the preceding claims to said keratin materials.
